(19) United States
(12) Patent Application Publication
(10) Pub. No.: US 2026/0014264 A1
(43) Pub. Date: Jan. 15, 2026

(54) SPECIFIC TOPOISOMERASE INHIBITOR, USE AS ANTIBODY DRUG CONJUGATE, AND PREPARATION METHOD THEREFOR

(71) Applicant: BIOBRICS LIFE SCIENCE (NANTONG) CO., LTD., Nantong, Jiangsu (CN)

(72) Inventors: Fangdao WANG, Shanghai (CN); Dong WANG, Shanghai (CN); Meng WANG, Shanghai (CN)

(73) Assignee: BIOBRICS LIFE SCIENCE (NANTONG) CO., LTD., Nantong, Jiangsu (CN)

(21) Appl. No.: 18/881,703

(22) PCT Filed: Jun. 28, 2023
(86) PCT No.: PCT/CN2023/103058

§ 371 Date: Jan. 6, 2025

(30) Foreign Application Priority Data
Jul. 5, 2022 (CN) 202210792928.2

(51) Int. Cl. A61K 47/68; A61P 35/00; C07D 491/22

(52) U.S. Cl. CPC A61K 47/68037; A61K 47/6849; A61K 47/6851; A61K 47/6853; A61K 47/6889; A61P 35/00; C07D 491/22

(57) ABSTRACT

A specific topoisomerase inhibitor, a use as an antibody drug conjugate, and a preparation method therefor, which relate to the technical field of medicinal chemistry. The inhibitor is a compound A or a tautomer, a mesomer, a racemate, an optical antipode, a diastereoisomer, or a mixture form thereof, or a pharmaceutically acceptable salt thereof; the structure of the compound A is such that the compound may also be further prepared to obtain an antibody drug conjugate, the antibody drug conjugate has good solubility and pharmaceutical properties, and the conjugation process does not result in precipitation, the antibody drug conjugate exhibits obvious in-vivo anti-tumor activity, and shows markedly stronger anti-tumor activity when compared with a control sample.

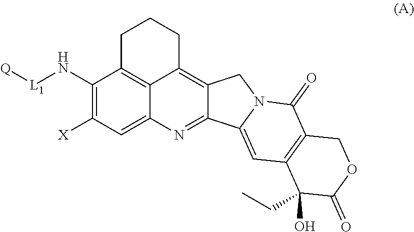

### FIELD OF INVENTION

The present invention relates to the specific topoisomerase inhibitor, the linker-payload conjugate used for an antibody drug conjugate, Preparation method for the related compounds and antibody drug conjugates, and their use in Preparation of drugs for treating cancer, is belong to the field of medicinal chemistry technology.

### PRIOR ARTS

The basic modules of antibody conjugated drugs include antibody, linker, and toxin molecules. By using antibodies to transport toxin molecules to tumor sites, tumor cells can be killed. Traditional toxin molecules are mostly highly active microtubule inhibitors or directly targeted DNA cytotoxic drugs, The significant toxic side effects limit the application of ADC.

Recently, Immunomidecs Inc. has developed a new ADC drug IMMU-132 using camptothecin compound as payload, which has shown good anti-tumor effects. Daiichi Sankyo Company Limited has also developed an ADC drug DS-820a with another camptothecin compound as payload, which has also shown good anti-tumor effects.

In the existing ADC technology, the main method is to modify the existing linker technology to connect camptothecin compounds with antibodies. Generally speaking, the ideal linker in ADC needs to meet the following requirements: firstly, ensure that small molecule drugs do not detach from antibodies in plasma, and the linker will break under appropriate conditions to rapidly release active small molecule drugs after entering cells; Secondly, the linker also needs to have good physicochemical properties in order to form conjugates with antibodies; thirdly, the linker should be easy to prepare in order to lay the foundation for large-scale production of ADCs. IMMU-132 uses pH sensitive linkers with poor stability, while DS-820a uses a tetrapeptide structure containing glycine-glycine-phenylalanine-glycine (GGFG) with good stability. The toxins released by the above-mentioned ADC drugs are SN38 and Dxd, both of which are Pgp substrates, and there are still issues such as tumor multidrug resistance.

Therefore, further development of camptothecin derivatives and ADC drugs with better efficacy and/or safety is still needed.

### CONTENT OF THE PRESENT INVENTION

To overcome the above-mentioned technical defects, the present invention provides an inhibitor compound or its tautomer, mesomer, racemate, optical antipode, diastereoisomer, or the mixture form thereof, or the pharmaceutically acceptable salt thereof, specifically, it comprises the structure shown in formula (A):

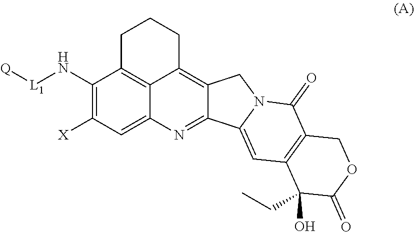

Wherein X is hydrogen and fluorine; Q is hydrogen or a group that can be conjugated with antibodies, L1 is a group that connects Q and the amino group of the drug.

Further, in the above technical solution, Q part is a group that can be conjugated with the thiol group on the antibody, selected from maleimide.

Further, in the above technical solution, L1 is a group that connects Q and the amino group of the drug, selected from

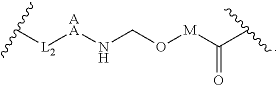

Wherein L2 is an optionally substituted C3-C7 alkylene, C3-C8 cyclic alkyl, optionally substituted diethylene glycol to octaethylene glycol acyl; AA is a peptide fragment composed of 2 to 4 amino acids; M is a methylene, C1-C6 alkyl, or cycloalkyl substituted methylene, trifluoromethyl substituted methylene, and C3-C6 cyclic alkyl.

Further, in the above technical solution, L1 is a group that connects Q and the amino group of the drug, selected from

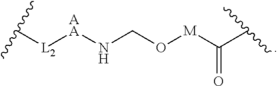

Wherein AA is a peptide fragment selected from ^{NH}-Phe-Lys-^{C═O}, ^{NH}-Val-Cit-^{C═O}, ^{NH}_Val-Ala-^{C═O}, ^{NH}-Phe-Cit-^{C═O}, ^{NH}-Gly-Val-^{C═O}, ^{NH}-Ala-Lys-^{C═O}, ^{NH}-Ala-Ala-Ala-^{C═O}, ^{NH}-Glu-Val-Ala-^{C═O}, ^{NH}-Glu-Val-Cit-^{C═O}, ^{NH}-Gly-Gly-Phe-Gly-^{C═O}.

Further, in the above technical solution, L1 is a group that connects Q and the amino group of the drug, selected from

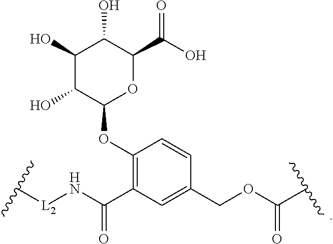

Wherein L2 is an optionally substituted C3-C7 alkylene, C3-C8 cyclic alkyl, optionally substituted diethylene glycol to octaethylene glycol acyl.

Further, in the above technical solution, the specific molecular structure of formula A is as follows:

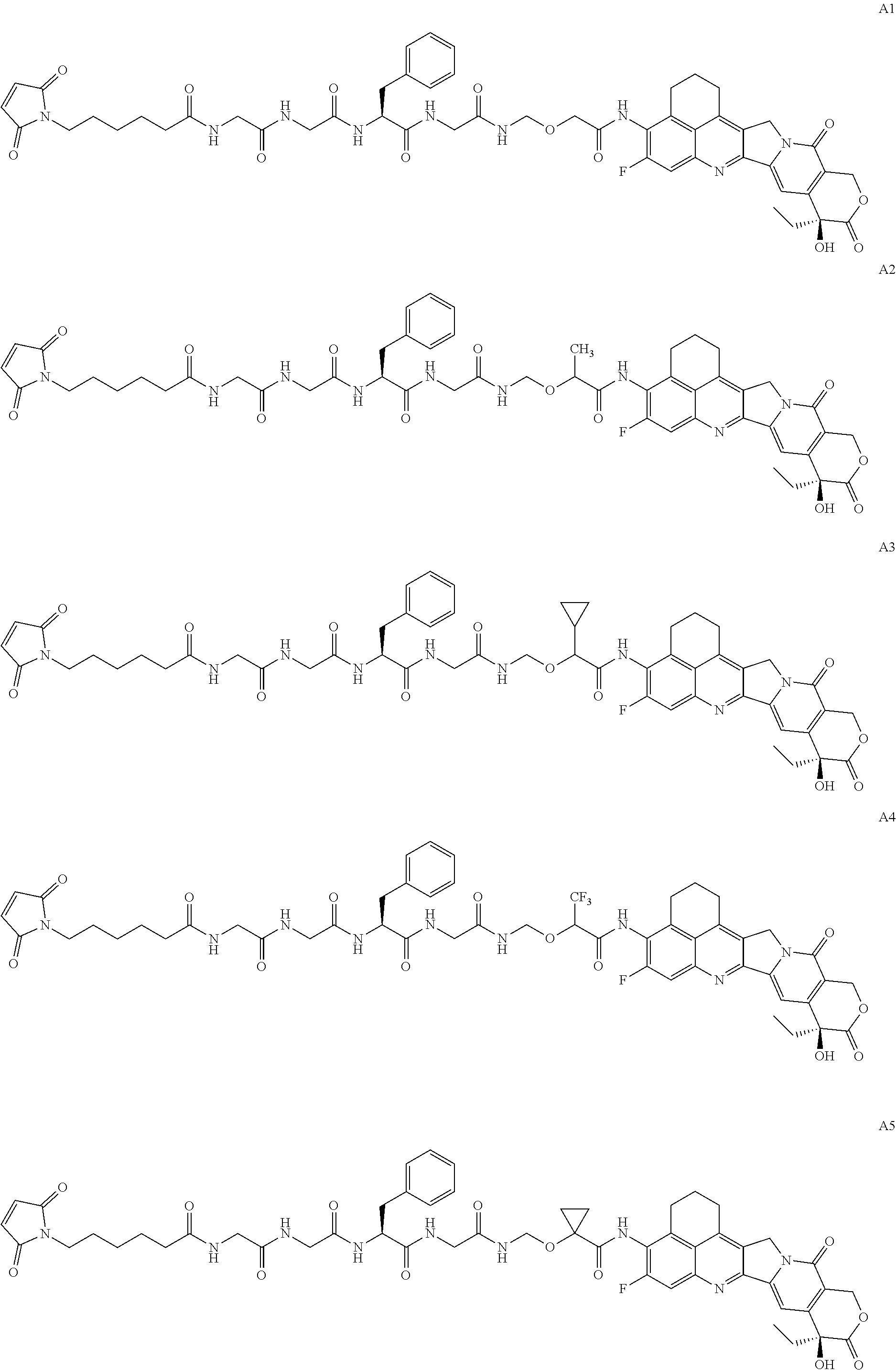

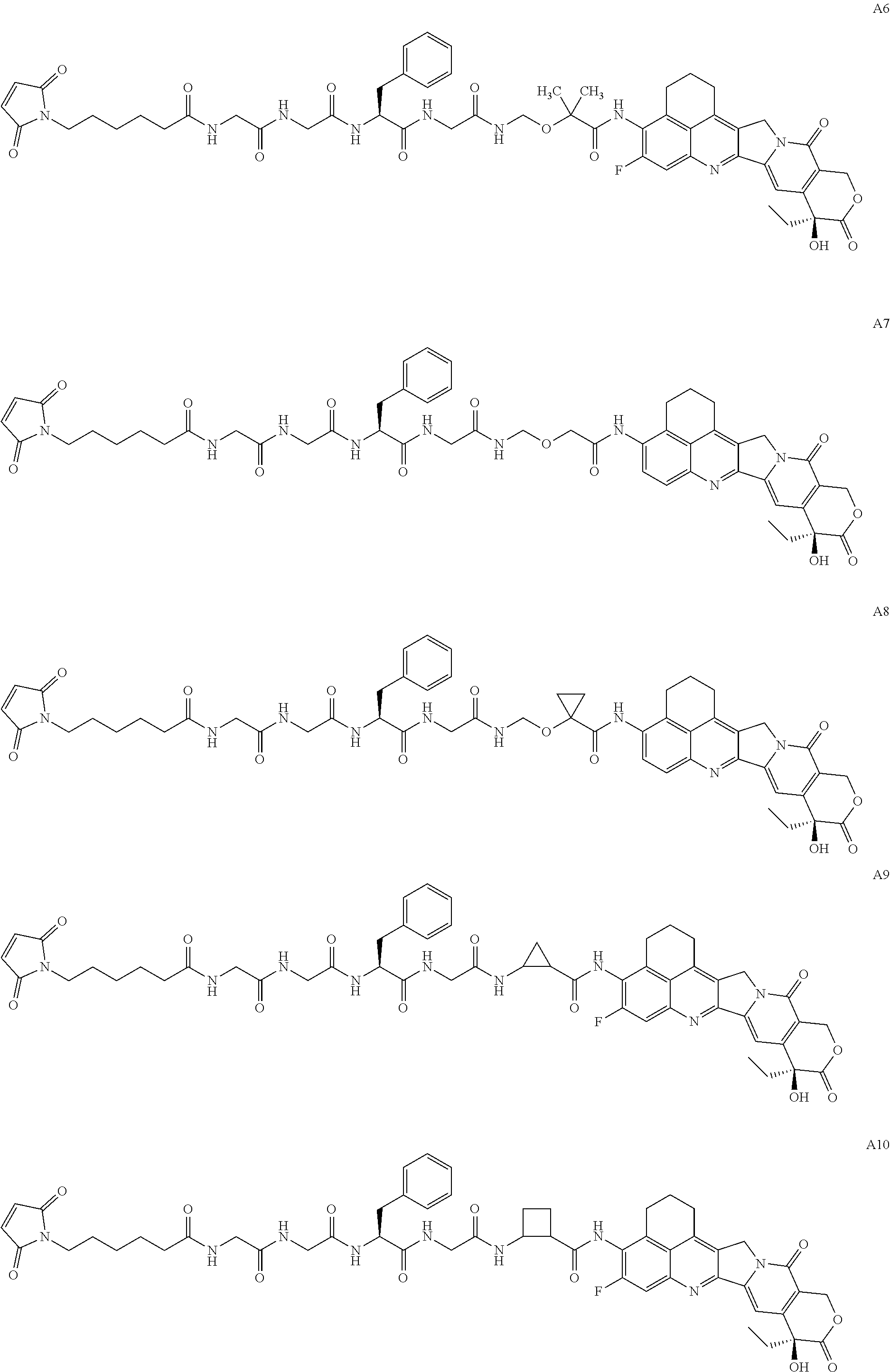

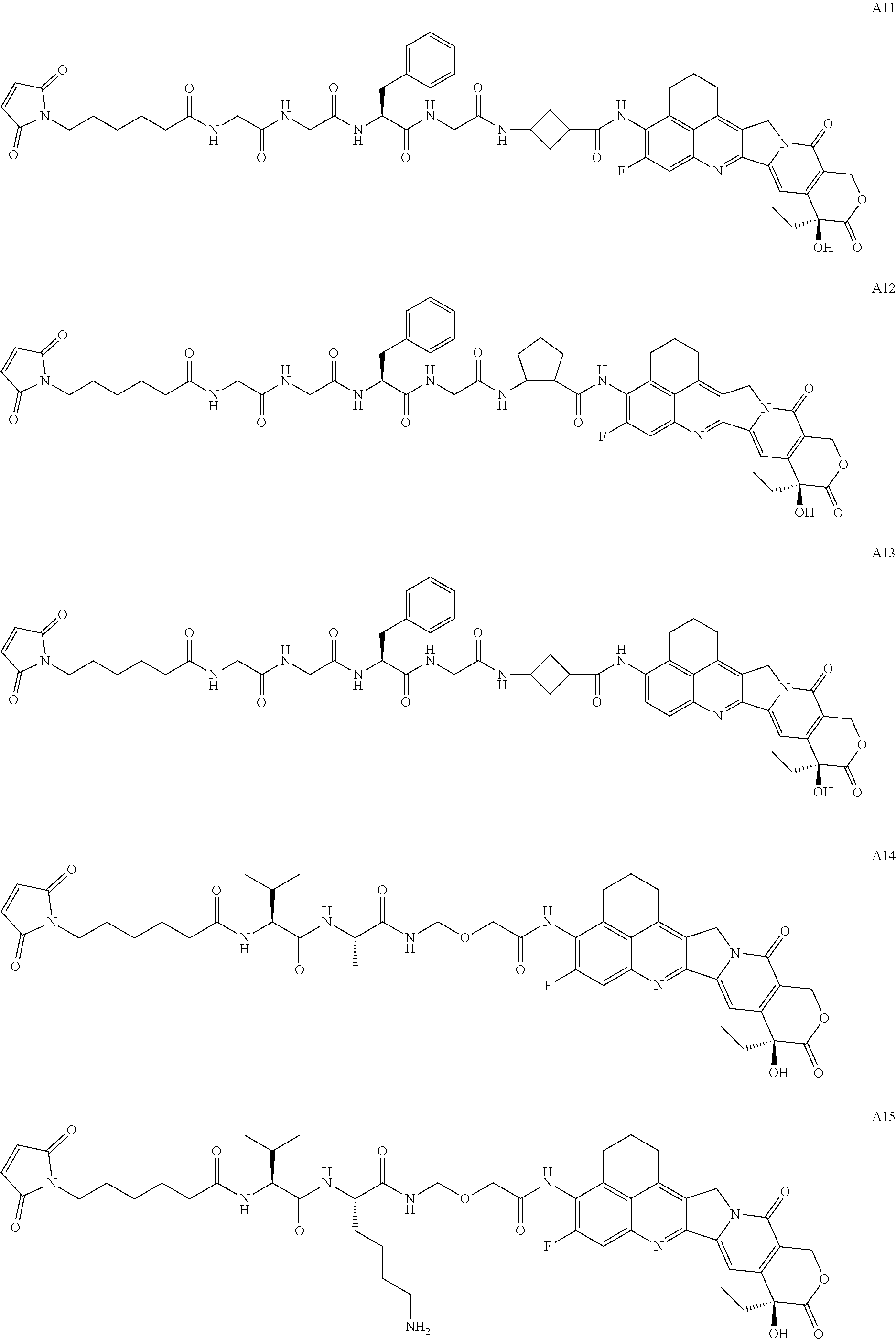

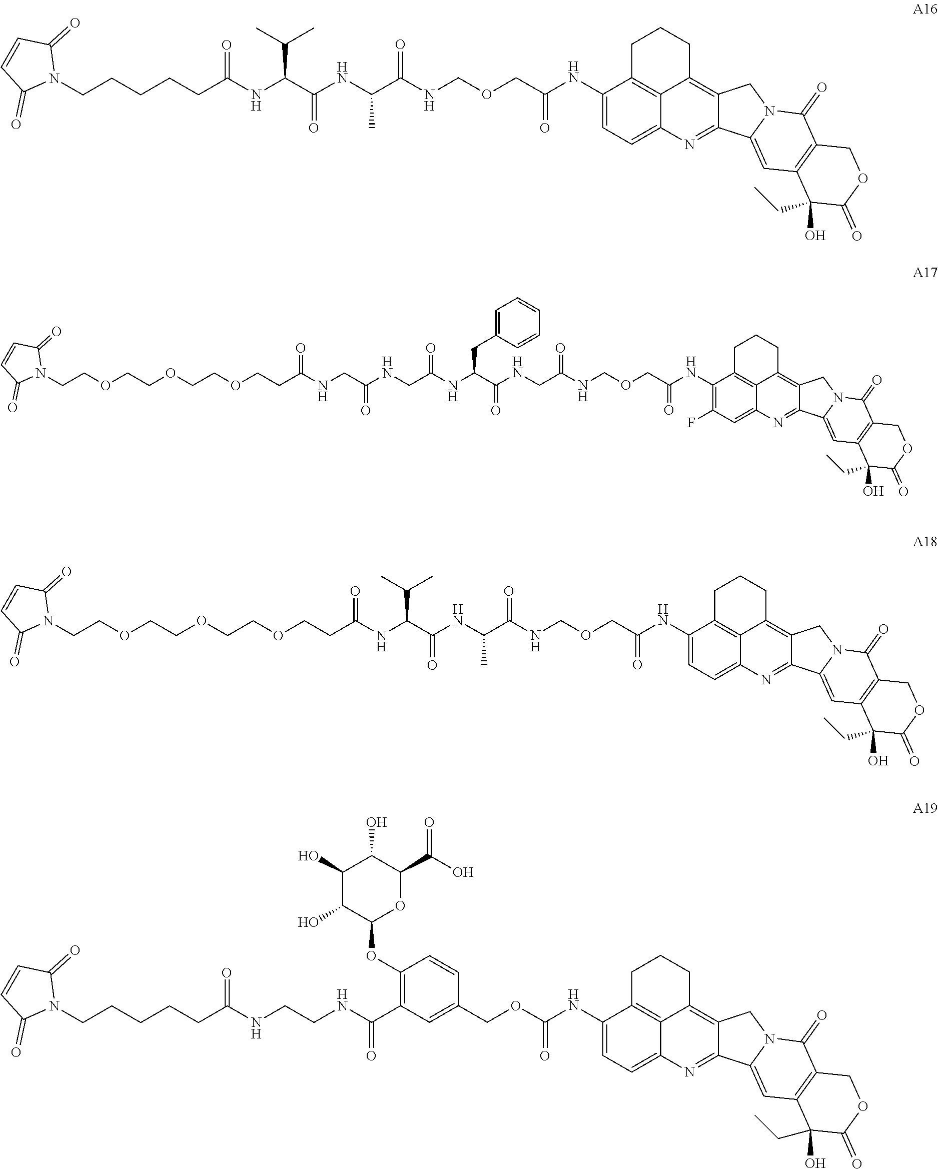

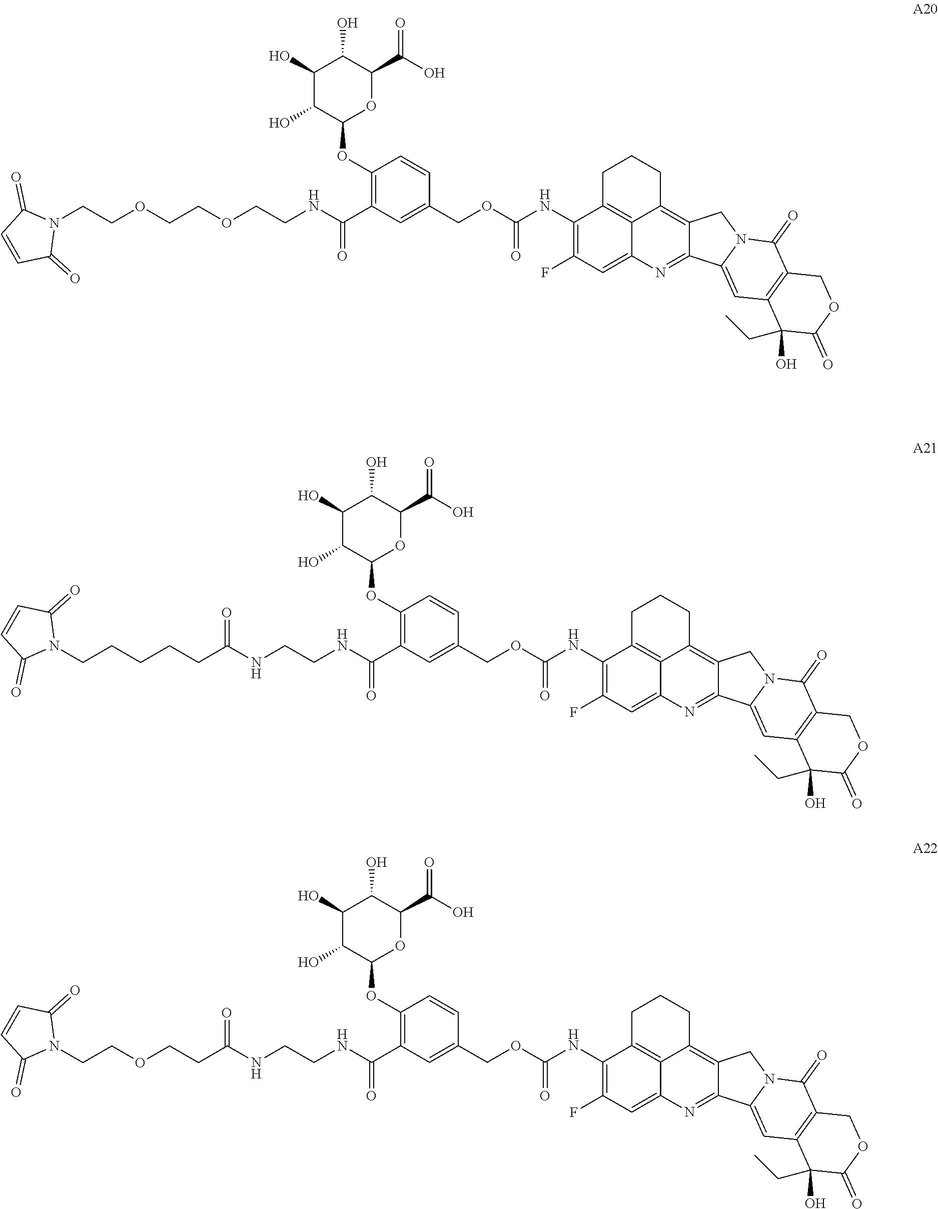 [SMILES: CC[C@@]1(O)C(=O)OCc2c1cc1n(c2=O)Cc2c-1nc1cc(F)c(NC(=O)OCc3ccc(O[C@@H]4O[C@H](C(=O)O)[C@@H](O)[C@H](O)[C@H]4O)c(C(=O)NCCOCCOCCN4C(=O)C=CC4=O)c3)c3c1c2CCC3 ; CC[C@@]1(O)C(=O)OCc2c1cc1n(c2=O)Cc2c-1nc1cc(F)c(NC(=O)OCc3ccc(O[C@@H]4O[C@H](C(=O)O)[C@@H](O)[C@H](O)[C@H]4O)c(C(=O)NCCNC(=O)CCCCCN4C(=O)C=CC4=O)c3)c3c1c2CCC3 ; CC[C@@]1(O)C(=O)OCc2c1cc1n(c2=O)Cc2c-1nc1cc(F)c(NC(=O)OCc3ccc(O[C@@H]4O[C@H](C(=O)O)[C@@H](O)[C@H](O)[C@H]4O)c(C(=O)NCCNC(=O)CCOCCN4C(=O)C=CC4=O)c3)c3c1c2CCC3]

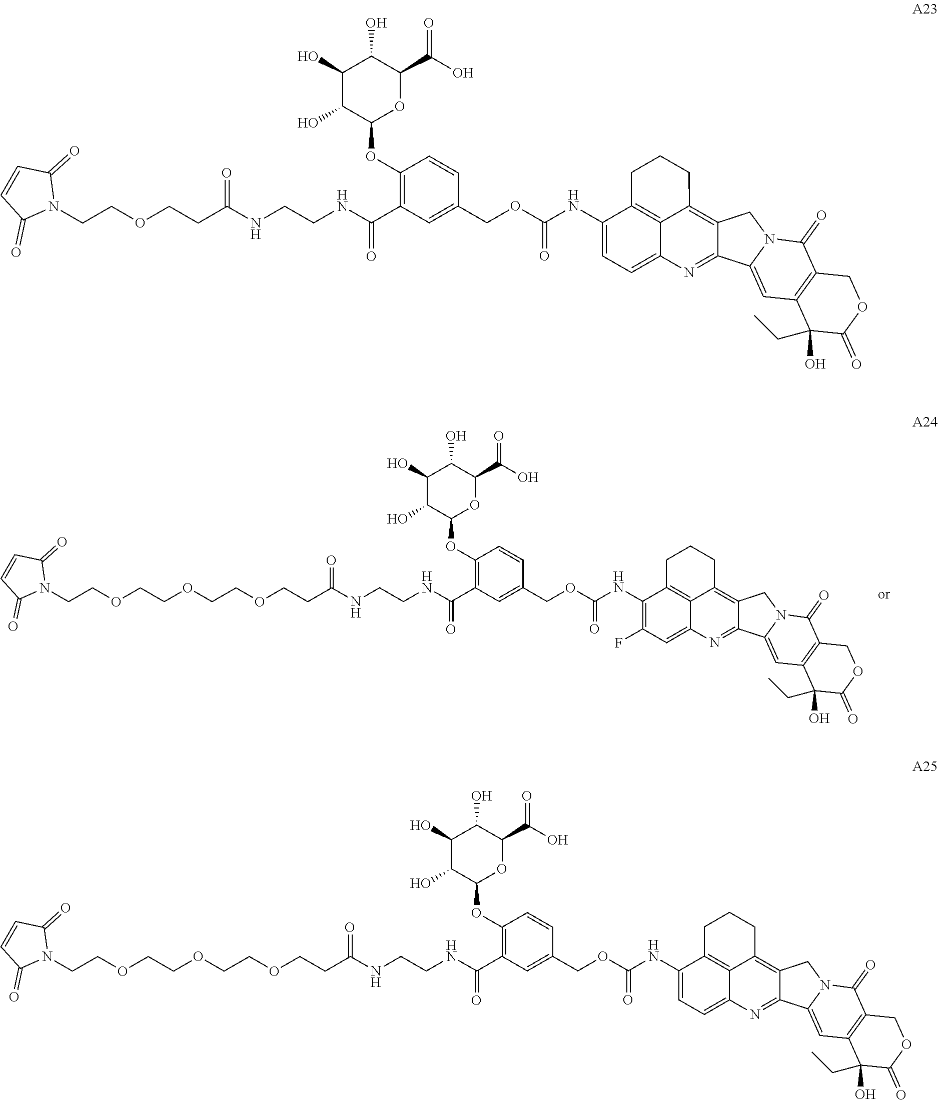 [SMILES: CC[C@@]1(O)C(=O)OCc2c1cc1n(c2=O)Cc2c-1nc1ccc(NC(=O)OCc3ccc(O[C@@H]4O[C@H](C(=O)O)[C@@H](O)[C@H](O)[C@H]4O)c(C(=O)NCCNC(=O)CCOCCN4C(=O)C=CC4=O)c3)c3c1c2CCC3 ; CC[C@@]1(O)C(=O)OCc2c1cc1n(c2=O)Cc2c-1nc1cc(F)c(NC(=O)OCc3ccc(O[C@@H]4O[C@H](C(=O)O)[C@@H](O)[C@H](O)[C@H]4O)c(C(=O)NCCNC(=O)CCOCCOCCOCCN4C(=O)C=CC4=O)c3)c3c1c2CCC3 ; "or" ; CC[C@@]1(O)C(=O)OCc2c1cc1n(c2=O)Cc2c-1nc1ccc(NC(=O)OCc3ccc(O[C@@H]4O[C@H](C(=O)O)[C@@H](O)[C@H](O)[C@H]4O)c(C(=O)NCCNC(=O)CCOCCOCCOCCN4C(=O)C=CC4=O)c3)c3c1c2CCC3]

The present invention provides an antibody drug conjugate or its tautomer, mesomer, racemate, optical antipode, diastereoisomer, or the mixture form thereof, or the pharmaceutically acceptable salt thereof. Specifically, it comprises the structure shown in formula (B):

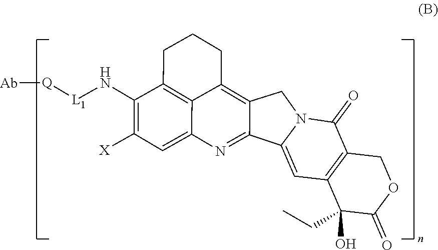

Wherein X is hydrogen and fluorine; Q is a group that can be conjugated with antibodies, L1 is a group that connects Q and the amino group of the drug, Ab is a ligand, n=1-8.

Further, in the above technical solution, Q part comprises the conjugated compound which is obtained by conjugating with the thiol group, selected from

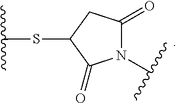 [SMILES: *SC1CC(=O)N(*)C1=O]

Further, in the above technical solution, L1 is a group that connects Q and the amino group of the drug, selected from

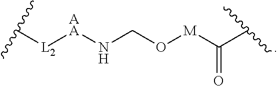

Wherein L2 is an optionally substituted C3-C7 alkylene, C3-C8 cyclic alkyl, optionally substituted diethylene glycol to octaethylene glycol acyl; AA is a peptide fragment composed of 2 to 4 amino acids; M is a methylene, C1-C6 alkyl, or cycloalkyl substituted methylene, trifluoromethyl substituted methylene, and C3-C6 cyclic alkyl.

Further, in the above technical solution, L1 is a group that connects Q and the amino group of the drug, selected from

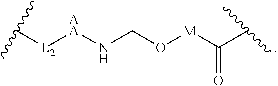

Wherein AA is a peptide fragment selected from ^{NH}-Phe-Lys-^{C═O}, ^{NH}-Val-Cit-^{C═O}, ^{NH}-Val-Ala-^{C═O}, ^{NH}-Phe-Cit-^{C═O}, ^{NH}-Gly-Val-^{C═O}, ^{NH}-Ala-Lys-^{C═O}, ^{NH}-Ala-Ala-Ala-^{C═O}, ^{NH}-Glu-Val-Ala-^{C═O}, ^{NH}-Glu-Val-Cit-^{C═O}, ^{NH}-Gly-Gly-Phe-Gly-^{C═O}.

Further, in the above technical solution, L1 is a group that connects Q and the amino group of the drug, selected from

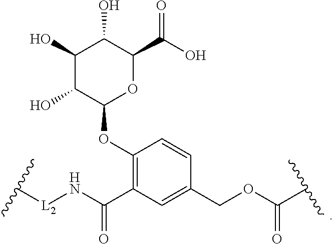

Wherein L2 is an optionally substituted C3-C7 alkylene, C3-C8 cyclic alkyl, optionally substituted diethylene glycol to octaethylene glycol acyl.

Further, in the above technical solution, the specific molecular structure of formula B is as follows:

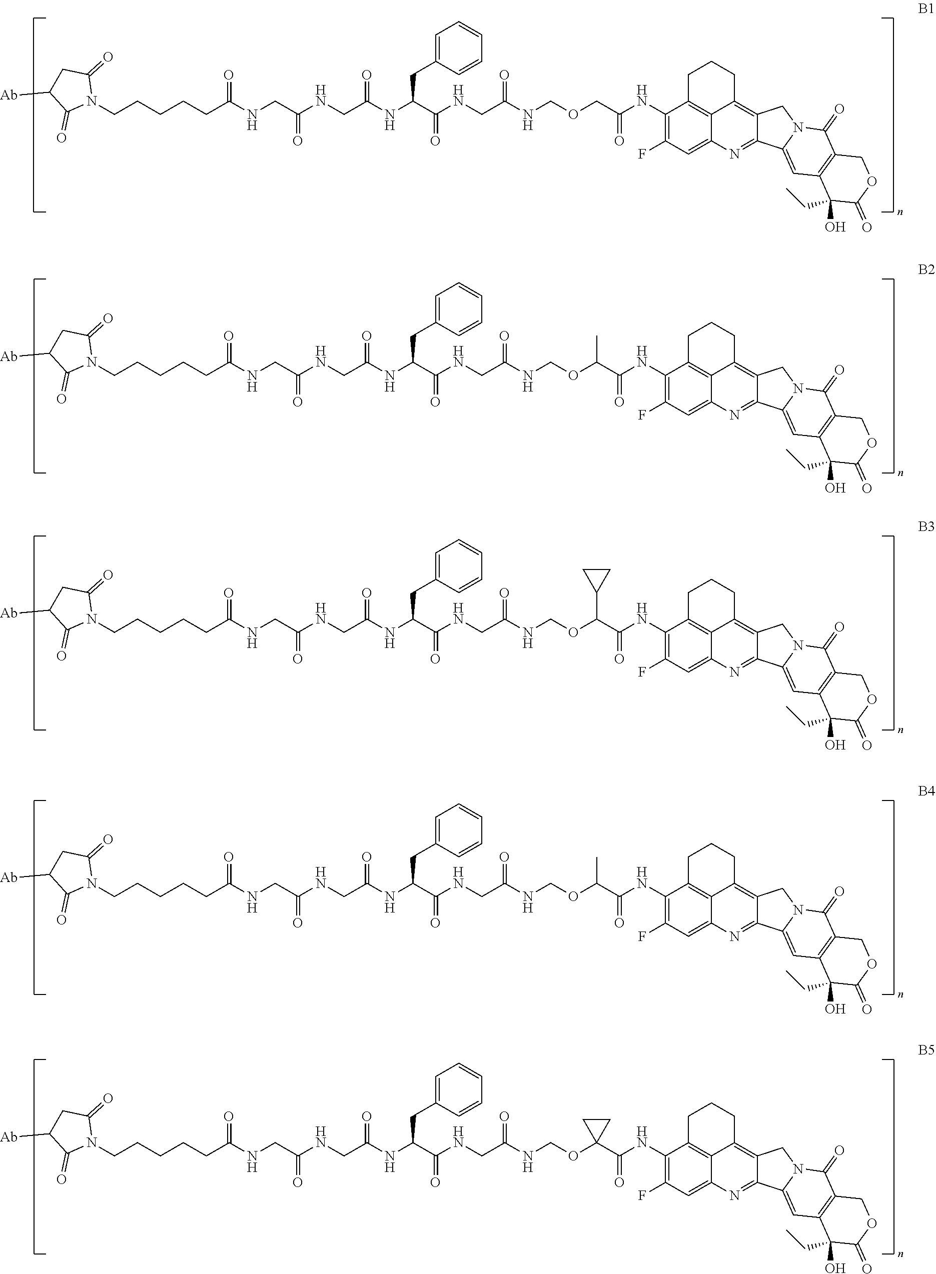

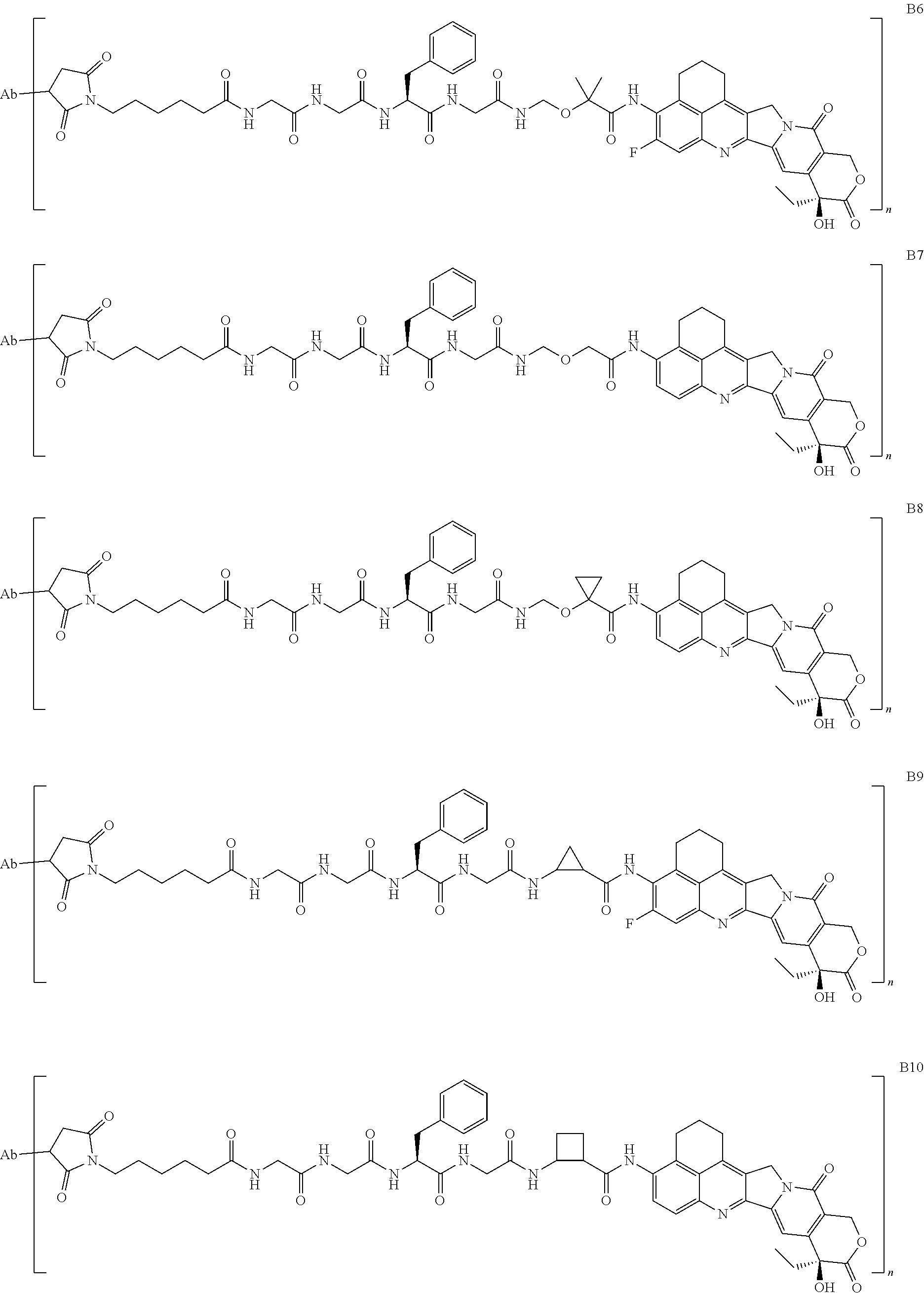

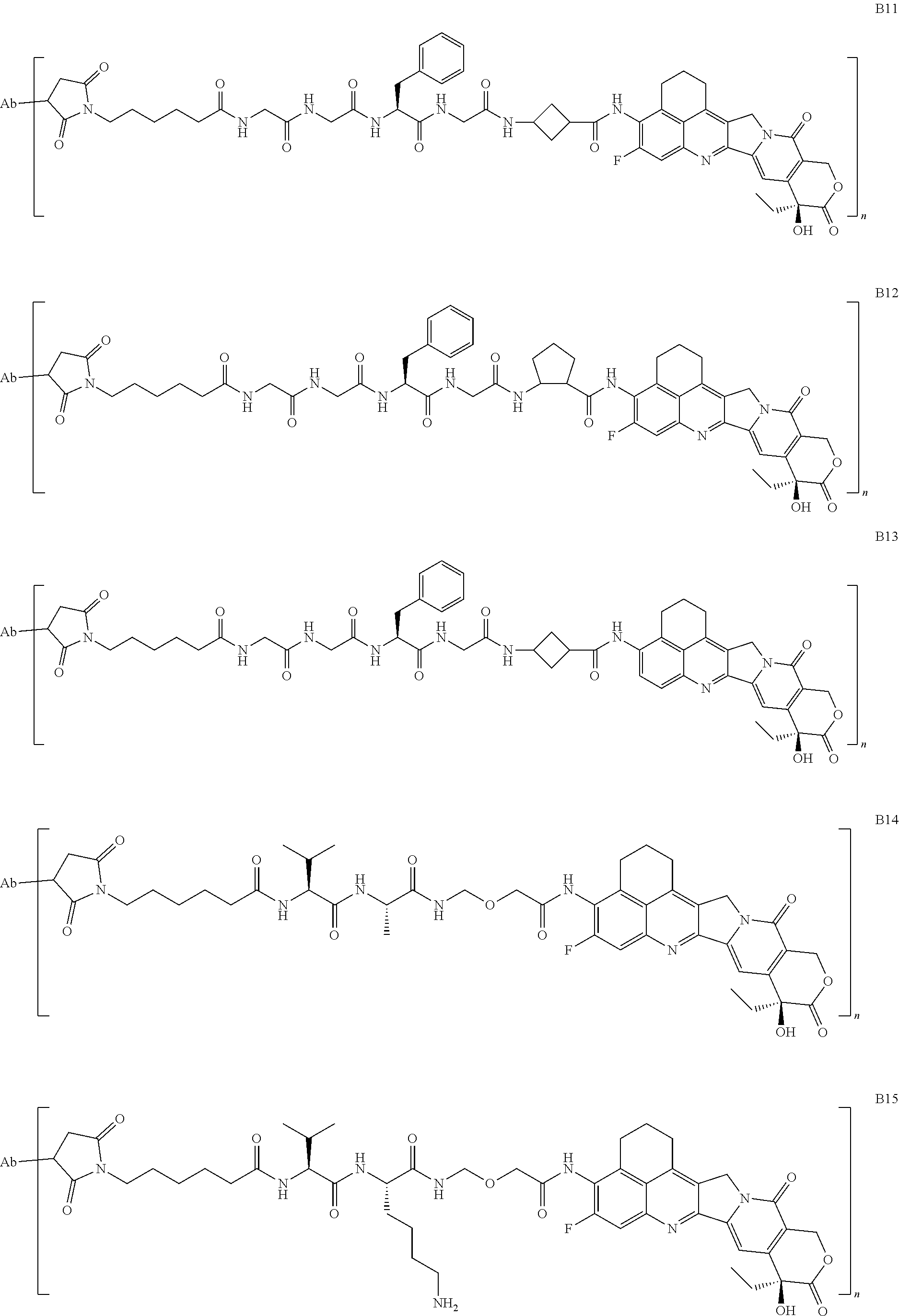

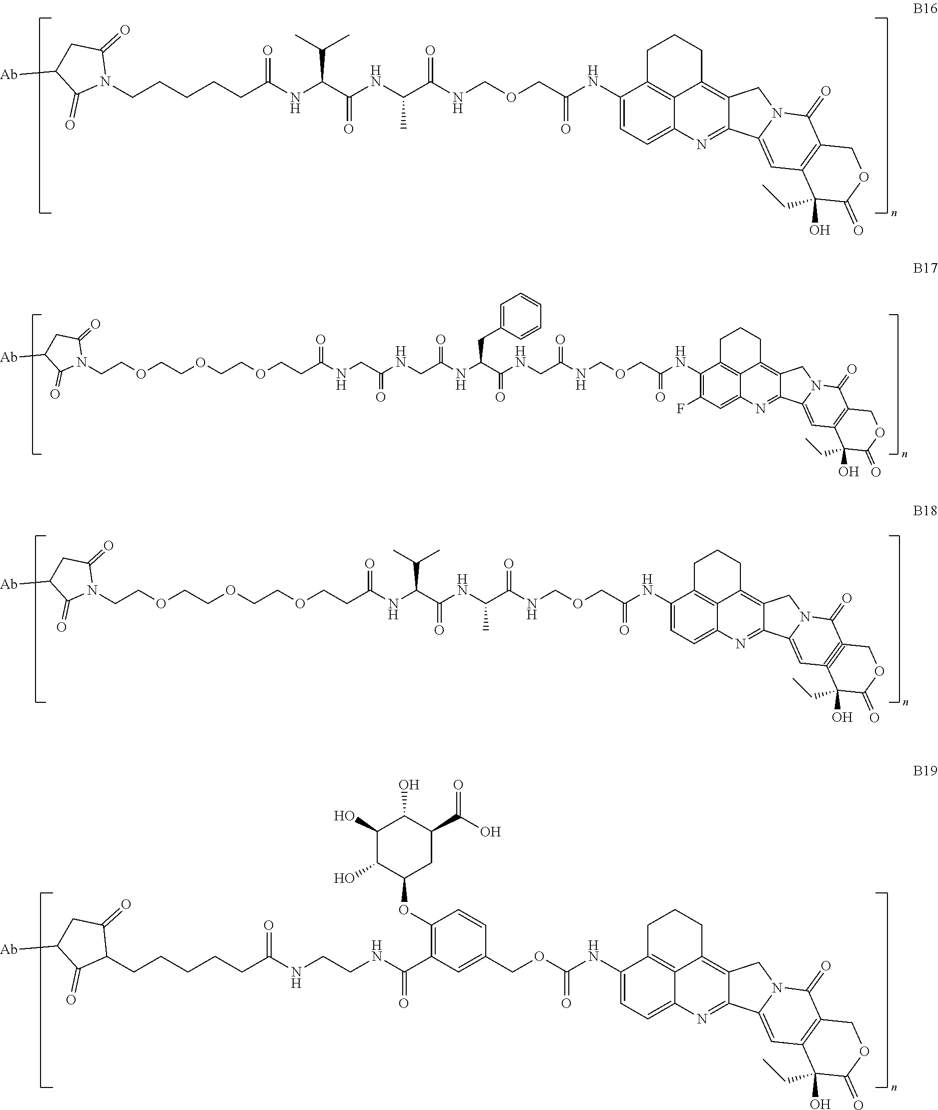

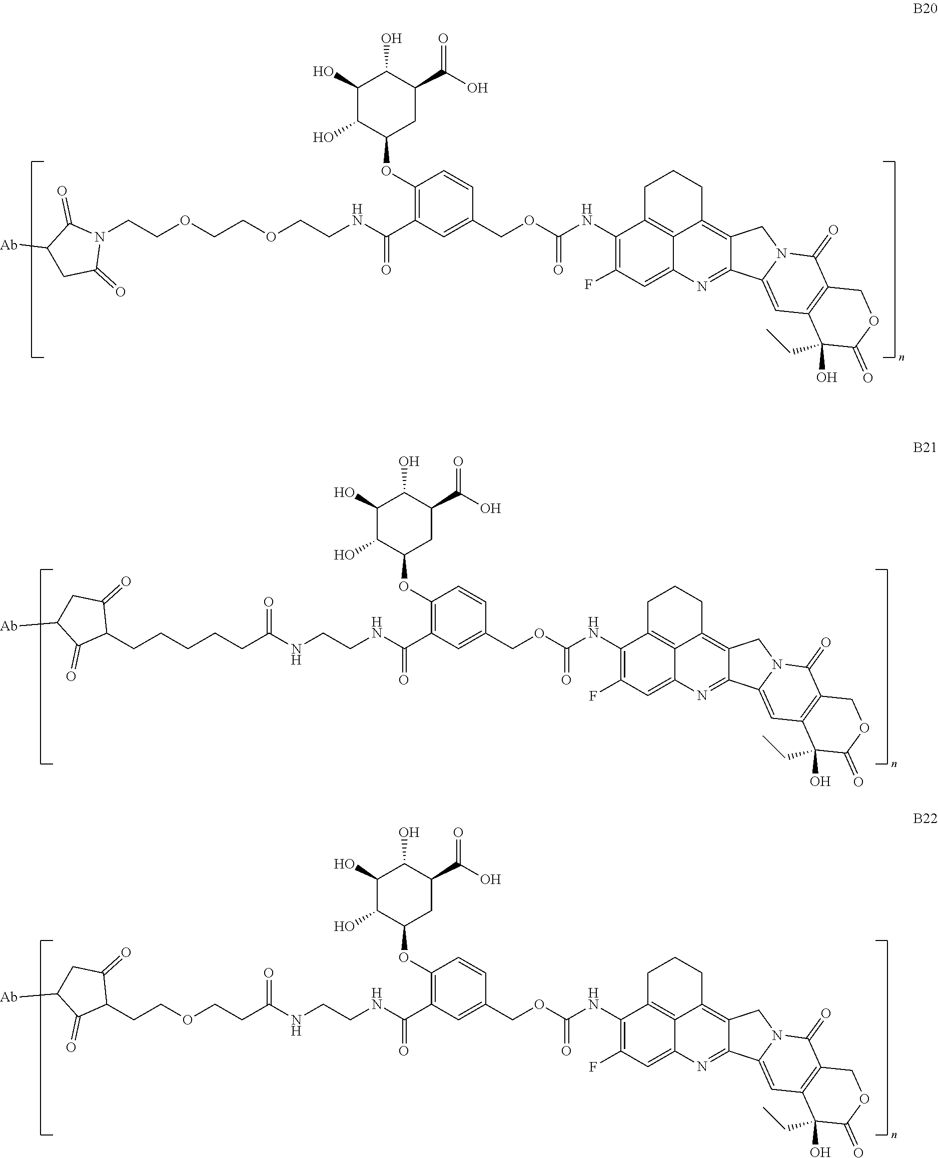

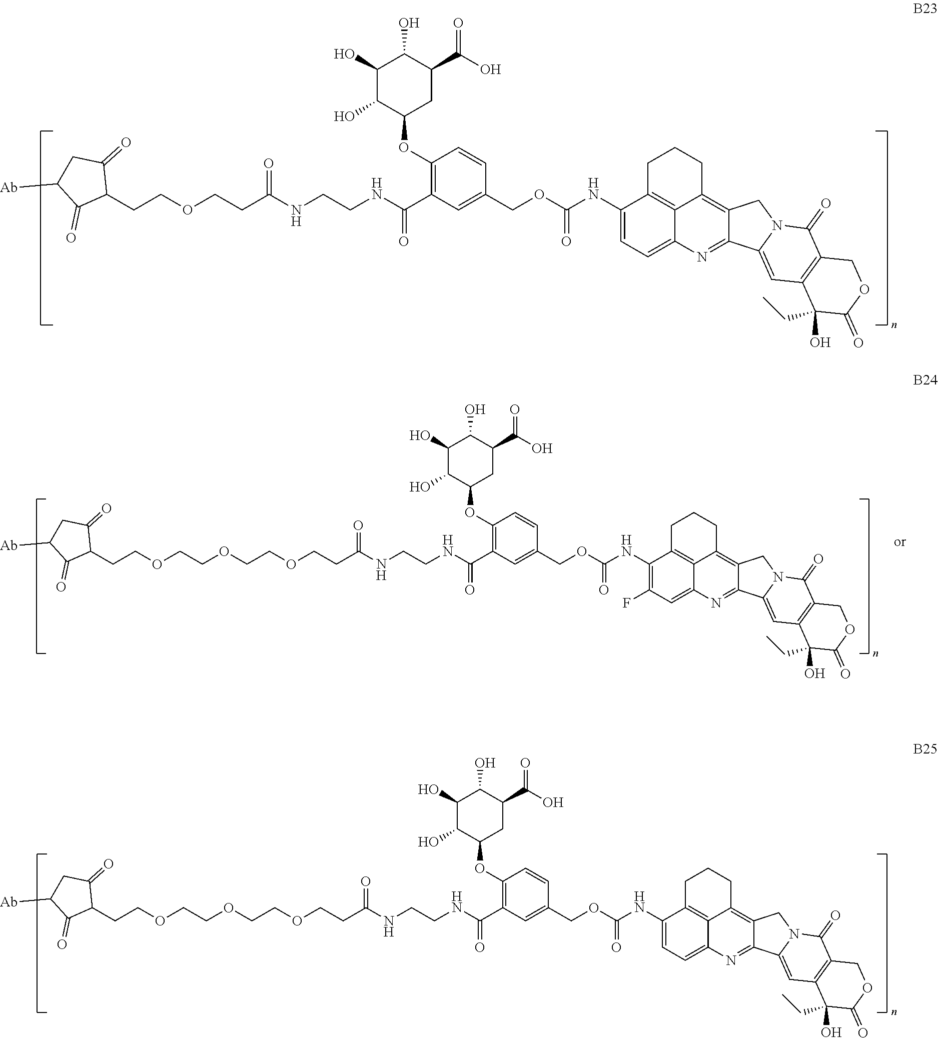

Wherein Ab is a ligand, n=1-8.

Further, in the above technical solution, Ab is selected from mouse derived antibodies, chimeric antibodies, humanized antibodies, or fully humanized antibodies.

Further, in the above technical solution, Ab comprises monoclonal antibodies.

Further, in the above technical solution, Ab comprises bispecific antibodies.

Further, in the above technical solution, the according antibody can bind to HER2, HER3, CD19, CD20, CD22, CD30, CD33, CD37, CD45, CD56, CD66e, CD70, CD74, CD79b, CD138, CD147, CD223, EpCAM, Mucin 1, STEAP1, GPNMB, FGF2, FOLR1, EGFR, EGFRvIII, Tissue factor, c-MET, FGFR, Nectin 4, AGS-16, Guanylyl cyclase C, Mesothelin, SLC44A4, PSMA, EphA2, AGS-5, GPC-3, c-KIT, ROR1, PD-L1, CD27L, 5T4, Mucin 16, NaPi2b, STEAP, SLITRK6, ETBR, BCMA, Trop-2, CEACAM5, SC-16, SLC39A6, Delta like protein 3, or Claudin 18.2 tumor associated antigens.

The present invention also provides a pharmaceutical composition comprises: (a) the above-mentioned antibody drug conjugates; And (b) pharmaceutically acceptable diluents, carriers, or excipients.

The present invention also provides the use of the above-mentioned antibody drug conjugates for preparation of therapeutic drugs for tumors.

The present invention also provides Preparation method of the above-mentioned antibody drug conjugates. Specifically, it comprises the following steps:
a. Reacting antibodies with reducing reagents in buffer solution to obtain reduced antibodies;
b. The linker-payload conjugate (A) is conjugated in a mixture of buffer solution and organic solvent with the reduced antibody obtained in the step a to obtain the antibody drug conjugate.

### DETAILED DESCRIPTION OF THE PREFERRED EMBODIMENT

The following specific embodiments illustrate the implementation of the present invention, and those familiar with this technology can easily understand other advantages and effects of the present invention from the disclosed content in this specification.

### Definitions

In the present invention, the term ‘pharmaceutically acceptable’ ingredient refers to a substance that is suitable for use in humans and/or animals without excessive adverse side effects (such as toxicity, irritation, and hypersensitivity), i.e. has a reasonable benefit/risk ratio.

In the present invention, the term ‘effective amount’ refers to the amount of a therapeutic agent used to treat, alleviate, or prevent a target disease or condition, or an amount that exhibits detectable therapeutic or preventive effects. The precise effective dosage for a certain object depends on the object's body shape and health condition, the nature and degree of the disease, as well as the selected therapeutic agent and/or combination of therapeutic agents. Therefore, specifying the exact effective amount in advance is useless. However, for a given condition, the effective dose can be determined using routine experiments, and clinical physicians can make judgments.

Unless otherwise specified, all compounds appearing in the present invention are intended to include all possible optical isomers, such as single chiral compounds, or mixtures of various chiral compounds (i.e. racemates). Among all compounds of the present invention, each chiral carbon atom can optionally be in the R or S configuration, or a mixture of R and S configurations.

As used in this article, the term ‘compound of the present invention’ refers to the compound shown in formula I. The term also includes various crystal forms, pharmaceutically acceptable salts, hydrates, or solvates of compounds of formula I.

As used in this article, the term ‘pharmaceutically acceptable salt’ refers to a salt formed by a compound of the present invention with an acid or base that is suitable for use as a drug. Pharmaceutically acceptable salts include inorganic salts and organic salts. A preferred type of salt is the salt formed between the compound of the present invention and an acid. Suitable acids for salt formation include but are not limited to inorganic acids such as hydrochloric acid, hydrobromic acid, hydrofluoric acid, sulfuric acid, nitric acid, phosphoric acid, as well as organic acids such as formic acid, acetic acid, propionic acid, oxalic acid, malonic acid, succinic acid, fumaric acid, maleic acid, lactic acid, malic acid, tartaric acid, citric acid, picric acid, methanesulfonic acid, benzenesulfonic acid, and benzenesulfonic acid; And acidic amino acids such as aspartic acid and glutamic acid.

Unless otherwise specified, the term “amino acid” used in this article is intended to include any conventional amino acid, such as aspartic acid, glutamic acid, cysteine, asparagine, phenylalanine, glutamine, tyrosine, serine, methionine (methionine), tryptophan, glycine, valine, leucine, alanine, isoleucine, proline, threonine, histidine, lysine, arginine.

When a trade name is used in this article, it is intended to include the product formulation of the trade name, its corresponding generic drug, and the active pharmaceutical component of the trade name product.

The term “antibody” in this article is used in its broadest sense and specifically covers monoclonal antibodies, polyclonal antibodies, dimers, multimers, multispecific antibodies (such as bispecific antibodies), and antibody fragments, as long as they exhibit the desired biological activity (Miller et al. (2003) Journal of Immunology 170:4854-4861). Antibodies can be mouse, human, humanized, chimeric, or derived from other species. Antibodies are proteins produced by the immune system that can recognize and bind to specific antigens (Janeway, C., Travers, P., Walport, M., Shlomchik (2001) ImmunoBiology, 5th Ed., Garland Publishing, New York). The target antigen generally has a large number of binding sites, also known as epitopes, recognized by CDRs of multiple antibodies. Antibodies that specifically bind to different epitopes have different structures. Therefore, an antigen can have more than one corresponding antibody. Antibodies include full-length immunoglobulin molecules or the immunologically active portion of full-length immunoglobulin molecules, which contain molecules that specifically bind to the target of interest, including but not limited to cancer cells or cells that produce autoimmune antibodies associated with autoimmune diseases. The immunoglobulin disclosed in this article can have any type (such as IgG, IgE, IgM, IgD, and IgA), class (such as IgG1, IgG2, IgG3, IgG4, IgA1, and IgA2), or subclass of immunoglobulin molecules. Immunoglobulin can come from any species. However, in one aspect, immunoglobulin is derived from humans, mice, or rabbits.

The ‘antibody fragment’ contains a portion of the full-length antibody, typically its antigen binding region or variable region. Examples of antibody fragments include Fab, Fab ‘, F (ab’) 2, and Fv fragments; Double antibody; Linear antibody; Microbody (Olafsen et al. (2004) Protein Eng. Design&Sel. 17 (4): 315-323); Fragment prepared from Fab expression library; Anti idiotypic (anti Id) antibodies; CDR (Complementary Determination Region); And any epitope binding fragment that binds to cancer cell antigens, viral antigens, or microbial antigens in an immune specific manner; Single chain antibody molecules; Multi specific antibodies formed from antibody fragments.

The antibody composing the antibody drug conjugate in the present invention should preferably maintain its antigen binding ability in its original wild state. Therefore, the antibodies in the present invention can, preferably with specificity, bind to antigens. The involved antigens include, for example, tumor associated antigens (TAA), cell surface receptor proteins and other cell surface molecules, cell survival regulatory factors, cell proliferation regulatory factors, molecules related to tissue growth and differentiation (such as known or predicted functional ones), lymphokines, cytokines, molecules involved in cell cycle regulation, molecules involved in angiogenesis, and molecules related to angiogenesis (such as known antibody binding antigens can be one or a subset of the above classifications, while other subsets include other molecules/antigens with special properties (compared to the target antigen).

The antibodies used in antibody drug conjugates include, but are not limited to, antibodies targeting cell surface receptors and tumor associated antigens. Such tumor associated antigens are well-known in the industry and can be prepared using well-known antibody preparation methods and information. In order to develop effective cellular level targets for cancer diagnosis and treatment, researchers aim to search for transmembrane or other tumor associated peptides. These targets can be specifically expressed on the surface of one or more cancer cells, with little or no expression on the surface of one or more non cancer cells. Usually, compared to the surface of non cancer cells, such tumor associated peptides are more overexpressed on the surface of cancer cells. Confirming such tumor related factors can greatly enhance the specific targeting characteristics of antibody for cancer therapy.

In this application, the term “ligand” typically refers to a macromolecular compound that can recognize and bind to antigens or receptors associated with target cells. The role of ligands can be to present drugs to target cell populations that bind to ligands, including but not limited to protein hormones, lectins, growth factors, antibodies, or other molecules that can bind to cells, receptors, and/or antigens. In this application, the ligand can be represented as Ab, and the ligand antigen forms a connecting bond with the connecting unit through heteroatoms on the ligand, which can be an antibody or its antigen binding fragment. The antibody can be selected from chimeric antibodies, humanized antibodies, whole human antibodies, or mouse antibodies; The antibody may be a monoclonal antibody. For example, the antibody can be an antibody targeting the following targets: HER2, HER3, B7H, TROP2, Claudin 18.2, CD30, CD33, CD70, EGFR, 5T4, AGS-16, ANGPTL4, ApoE, CD19, CTGF, CXCR5, FGF2, MCPT8, MF12, MS4A7, NCA, Sema5b, SLITRK6, STC2, TGF, 0772P, ST4, ACTA2, ADGRE1, AG-7, AIF1, AKRIC1, AKR1C2, ASLG659, Axl, B7H3, BAFF-R, BCMA, BMPRIB, BNIP3, CIQA, CIQB, CA6, CADM1, CCD79b, CCL5, CCR5, CCR7, CD11c, CD123, CD138, CD142, CD147, CD166, CD19, CD22, CD21, CD20, CD205, CD22, CD223, CD228, CD25, CD30, CD33, CD37, CD38, CD40, CD45, CD45 (PTPRC), CD46, CD47, CD49D (ITGA4), CD56, CD66e, CD70, CD71, CD72, CD74, CD79a, CD79b, CDS0, CDCP1, CDH11, CD11b, CEA, CEACAMS, c-Met, COL6A3, COL7A1, CRIPTO, CSFIR, CTSD, CTSS, CXCL11, CXCL10, DDIT4, DLL3, DLL4, DR5, E16, EFNA4, EGFR, EGFRVIII, EGLN, EGLN3, EMR2, ENPP3, EpCAM, EphA2, EphB2R, ETBR, FcRH2, FcRHI, FGFR2, FGFR3, FLT3, FOLR-ALPHA, GD2, GEDA, GPC-1, GPNMB, GPR20, GZMB, HER2, HER3, HLA-DOB, HMOX1, IFI6, IFNG, IGF-1R, IGFBP3, IL-13R, IL-2, IL20Ra, IL-3, IL-4, IL-6, IRTA2, KISSIR, KRT33A, LIV-1, LOX, LRP-1, LRRC15, LUM, LY64, LY6E, Ly86, LYPD3, MDP, MMP10, MMP14, MMP16, MPF, MSG783, MSLN, MUC-1, NaPi2b, Napi3b, Nectin-4, NOG, P2X5, pAD, P-Cadherin, PDGFRA, PDK1, PD-L1, PFKFB3, PGF, PGK1, PIK3AP1, PIK3CD, PLOD2, PSCA, PSCAhlg, PSMA, PTK7, P-cadherin, RNF43, NaPi2b, ROR1, ROR2, SERPINE1, SLC39A6, SLTRK6, STAT1, STEAP1, STEAP2, TCF4, TENB2, TGFB1, TGFB2, TGFBR1, TNFRSF21, TNFSF9, Trop-2, TrpM4, Tyro7, UPKIB, VEGFA, WNTSA, epidermal growth factor, short proteoglycan, mesothelin, sodium phosphate cotransporter 2B, sealing protein 18.2, inner skin receptor, mucin (such as mucin 1 and mucin 16), guanylate cyclase C, integrin a4p7, integrin a5p6, trophoblast cell glycoprotein or tissue factor.

Enzyme unstable linkers, such as peptide linkers, can better control drug release. Peptide linkers can be effectively cleaved by proteases within the lysosome, such as cathepsin B or fibrinolytic enzymes (which have increased levels in some tumor tissues). This peptide linkage is considered to be very stable in plasma circulation, as inappropriate pH values outside the cell and serum protease inhibitors often render proteases inactive. Due to its high plasma stability and good intracellular cleavage selectivity and effectiveness, enzyme unstable linkers are widely used as breakable linkers for antibody drug conjugates. Typical enzyme unstable linkers include Val-Kit (VC), Phe-Lys, and others.

The self-releasing linker is generally embedded between the breakable linker and the active drug, or is itself a part of the breakable linker. The mechanism of action of the self-releasing linker is that when the breakable linker is broken under appropriate conditions, the self-releasing linker can spontaneously undergo structural rearrangement, thereby releasing the active drug which is connected to it. Common self-releasing linkers include para-aminobenzyl alcohol (PAB) and β-glucuronide.

### Preparation Method of Antibody Drug Conjugate

The disulfide bonds between antibody chains are reduced to produce a total of 8 thiol groups; conjugating the substituted maleamide-linker-drug conjugates with the reduced antibody thiol groups generates the corresponding antibody drug conjugate.

Dilute the antibody stock solution with reaction buffer solution to 2-10 mg/mL, add 6.0-20 times excess molar ratio of tris (2-carboxyethyl) phosphine hydrochloride (TCEP), or 140-200 times excess molar ratio of dithiothreitol (DTT), and stir the reaction solution at 25° C. for 2-4 hours; The above mentioned reaction buffer solution includes 50 mM potassium dihydrogen phosphate sodium hydroxide (KH2PO4 NaOH)/150 mM sodium chloride (NaCl)/1 mM diethylenetriaminepentaacetic acid (DTPA), pH=6-9; 50 mM disodium hydrogen phosphate citric acid/150 mM sodium chloride (NaCl)/1 mM diethylenetriaminepentaacetic acid (DTPA), pH=6-9; 50 mM boric acid borax/150 mM sodium chloride (NaCl)/1 mM diethylenetriaminepentaacetic acid (DTPA), pH=6-9; 50 mM histidine sodium hydroxide/150 mM sodium chloride (NaCl)/1 mM diethylenetriaminepentaacetic acid (DTPA), pH=6-9, and PBS/1 mM diethylenetriaminepentaacetic acid (DTPA), pH=6-9.

Cool the above reaction solution to 0-10° C. If TCEP reduction is used, the maleimide compound can be added directly without purification (10 mg/mL pre dissolved in dimethylsulfoxide (DMSO), dimethylformamide (DMF) or diethylacetamide (DMA)), and ensure that the volume proportion of organic solvents in the reaction solution does not exceed 15%. Stir the coupling reaction at 10-25° C. for 2 hours. If DTT reduction is used, excess DTT needs to be removed through a desalination column or ultrafiltration after the reduction reaction is completed, and then the substituted maleimide compound is added for conjugation.

The conjugate reaction mixture was filtered and purified with sodium succinate/150 mM NaCl buffer or histidine acetic acid/sucrose gel using a desalination column, and the peak samples were collected according to the UV280 ultraviolet absorption value, or ultrafiltration several times. Then, the bacteria were removed through a filtration device with a pore size of 0.22 microns, store at −80° C.

The drug antibody conjugate ratio (DAR8) of the obtained antibody drug conjugate is relatively uniform. The different drug-linker compounds described in this patent can produce antibody drug conjugates with certain differences in product uniformity. To obtain samples with better uniformity, the following methods can be further used for separation and purification, including but not limited to hydrophobic interaction chromatography (HIC), molecular exclusion chromatography (SEC), and ion exchange chromatography (IEC).

### Drug Combination and Administration Method

The antibody drug conjugate provided by the present invention can target specific cell populations, bind to cell surface specific proteins (antigens), and release drugs in active form into cells through endocytosis or drug infiltration of the conjugate. Therefore, the antibody drug conjugate of the present invention can be used to treat target diseases, and the above-mentioned antibody drug conjugate can be administered to subjects (such as humans) in therapeutic effective amounts through appropriate routes. The subjects in need of treatment may be patients at risk or suspected of having symptoms related to the activity or expression levels of specific antigens. Such patients can be identified through routine physical examinations.

Conventional methods, known to ordinary technicians in the medical field, can be used to administer drug combinations to subjects, depending on the type of disease to be treated or the site of the disease. The composition can also be administered by other conventional routes, such as oral administration, parenteral administration, inhalation of spray, local, rectal, nasal, oral, vaginal or implantation. The term ‘parenteral’ used in this article includes subcutaneous, intradermal, intravenous, intramuscular, intra-articular, intra-arterial, synovial, sternal, intrathecal, intralesional, and intracranial injection or infusion techniques. In addition, it can be applied to the theme of injectable or biodegradable materials and methods through injectable depot routes, such as using 1-, 3-, or 6-month depots.

The injection composition can contain various carriers such as vegetable oil, dimethylacetamide, dimethylformamide, ethyl lactate, ethyl carbonate, isopropyl myristate, ethanol, polyols (glycerol, propylene glycol, liquid polyethylene glycol, etc.). For intravenous injection, water-soluble antibodies can be administered by intravenous infusion of drug formulations containing antibodies and physiologically acceptable excipients. Physiologically acceptable excipients may include, for example, 5% glucose, 0.9% saline, Ringer's solution, or other suitable excipients. Intramuscular preparations, such as sterile preparations of antibodies in a suitable soluble salt form, can dissolve and administer pharmaceutical excipients such as water exchange injections, 0.9% saline, or 5% glucose solutions.

When using the antibody drug conjugate of the present invention for treatment, it can be delivered through conventional methods in this field. For example, it can be introduced into cells by using liposomes, hydrogels, cyclodextrins, biodegradable nanocapsules, or bioadhesive microspheres. Alternatively, the nucleic acid or vector can be delivered locally through direct injection or by using an infusion pump. Other methods include the use of various transport and carrier systems through the use of conjugates and biodegradable polymers.

The pharmaceutical composition of the present invention contains a safe and effective amount of the antibody drug conjugate of the present invention and a pharmaceutically acceptable carrier. This type of carrier includes (but is not limited to): saline solution, buffer solution, glucose, water, glycerol, ethanol, and their combinations. Usually, drug formulations should match the mode of administration, and the drug combination of the present invention can be prepared in solution form, such as using physiological saline or aqueous solutions containing glucose and other adjuvants, prepared by conventional methods. The pharmaceutical composition should be manufactured under sterile conditions. The dosage of the active ingredient is the therapeutic effective amount.

The effective amount of the antibody drug conjugate described in the present invention may vary depending on the mode of administration and the severity of the disease to be treated. The selection of the optimal effective dosage can be determined by ordinary technical personnel in this field based on various factors (such as through clinical trials). The factors mentioned include but are not limited to: pharmacokinetic parameters of the bifunctional antibody conjugate, such as bioavailability, metabolism, half-life, etc; The severity of the disease to be treated, the patient's weight, the patient's immune status, and the route of administration. Generally, when the antibody drug conjugate of the present invention is administered daily at a dose of about 0.0001 mg/50 mg/kg animal body weight (preferably 0.001 mg-10 mg/kg animal body weight), satisfactory results can be obtained. For example, due to the urgent need for treatment, separate doses can be given several times a day, or the dose can be reduced proportionally.

The dosage forms of the compounds of the present invention for local administration include ointments, powders, patches, sprays, and inhalants. The active ingredient is mixed with physiologically acceptable carriers and any preservatives, buffering agents, or necessary propellants under sterile conditions.

The compound of the present invention can be administered alone or in combination with other pharmaceutically acceptable therapeutic agents.

When using a pharmaceutical composition, a safe and effective amount of the compound of the present invention is applied to a mammal in need of treatment (such as a human), where the dosage at the time of administration is the effective dosage considered in pharmacy. For a person weighing 60 kg, the daily dosage is usually 1-2000 mg, preferably 5-500 mg. Of course, the specific dosage should also consider factors such as the route of administration and the patient's health condition, which are within the skill range of skilled physicians.

The present invention will be further explained in conjunction with specific embodiments. It should be understood that these embodiments are only used to illustrate the present invention and not to limit the scope of the present invention. The experimental methods without specific conditions specified in the following examples are usually carried out under conventional conditions or conditions recommended by the manufacturer. Unless otherwise specified, percentages and portions are calculated by weight.

### Example 1: Preparation of Compounds

### 1. Preparation of Compound A1

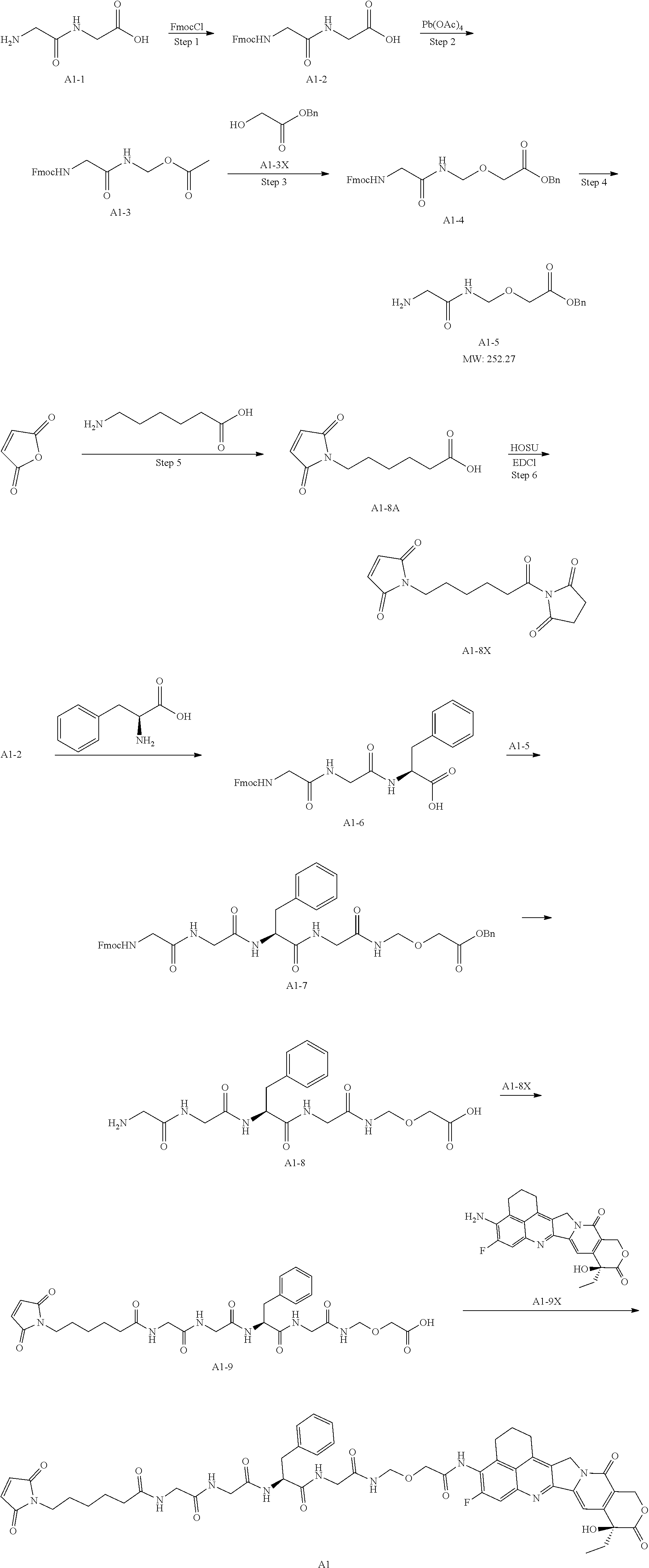

### Step 1

To the solution of diglycoside A1-1 (2.0 g, 15.2 mmol) and Fmoc-Cl (4.7 g, 18.2 mmol) in 1,4-dioxane (20 mL) was added 1M Na₂CO₃ aqueous solution (18 mL) dropwise at 0˜5° C. After the addition was completed, the reaction mixture was stirred at room temperature for 2 h. And then adjusted pH ˜2 with about 30 mL of 1M HCl at 0˜5° C. The resulting mixture was extracted with ethyl acetate (100 mL*3). The combined organic phase was concentrated under reduced pressure to give 6.0 g crude product as off-white solids. The crude product was scattered in MTBE (19 mL) and stirred at room temperature for 20 min. The solid was collected by filtration and dried under reduced pressure to give A1-2 (4.3 g, yield: 81.3%). MS(ESI)(m/z): 355 ([M+H]⁺).

### Step 2

To the solution of A1-2 (29.0 g, 81.9 mmol) and acetic acid (90 mL) in THF (450 mL) was added Pb(OAc)₄ (60.0 g, 135.4 mmol) at 40° C. under N₂ atmosphere, the mixture was heated to reflux for 16 h. After cooling down to room temperature, the reaction mixture was filtered through celite. After washing the filter cake with EA, the filtrate was concentrated and purified by silica gel column to give A1-3 (25.0 g, yield: 82.8%). MS(ESI)(m/z): 391 ([M+Na]⁺).

### Step 3

The solution of A1-3 (9.8 g, 26.6 mmol), A1-3X (17.6 g, 106.0 mmol) and PPTS (1.3 g, 5.2 mmol) in DCM (112 mL) was refluxed at 45° C. for 2 h under N₂ atmosphere. The reaction mixture was concentrated and purified by silica gel column to A1-4 (5.4 g, yield: 43.2%), MS(ESI)(m/z): 497 ([M+Na]⁺).

### Step 4

The solution of A1-4 (2.8 g, 5.9 mmol) and DBU (0.9 g, 5.9 mmol) in DMF (14 mL) was stirred at room temperature for 3-4 h under N₂ atmosphere. The reaction mixture was directly used in next step without further purification as a solution of A1-5 in DMF.

### Step 5

The solution of maleic anhydride (3.5 g, 36 mmol) and 6-aminocaproic acid (3.9 g, 30 mmol) in glacial acetic acid (39 mL) was stirred at 120° C. for 4 h. After cooling down to room temperature. the reaction solution was concentrated under reduced pressure to remove most of glacial acetic acid. The residua was diluted with EA, washed with water and brine, concentrated under reduced pressure to dryness. The residua was scattered in water (50 mL). After stirred at room temperature for 1 h, the suspension was filtered, the filter was dried under reduced pressure at 50° C. to give A1-8A (4.5 g, yield: 71.0%).

### Step 6

The suspension of A1-8A (4.5 g, 21.3 mmol), HOSU (2.7 g, 23.4 mmol), and EDCI (4.9 g, 25.6 mmol) in MeCN (45 mL) was stirred at room temperature for 16 h. After removing MeCN under reduced pressure, the residua was diluted with the sat. NaHCO₃ aqueous solution (50 mL), and extracted with DCM. The combined organic phase was concentrated and purified by silica gel column to give A1-8X (5.0 g, yield: 79.8%). MS(ESI)(m/z): 315.3 ([M+Na]⁺).

### Step 7

The solution of L-phenylalanine (5.2 g, 31.5 mmol), A1-2 (15.8 g, 44.6 mmol) and TEA (3.7 g, 36.5 mmol) in MeCN (56 mL) and H₂O (56 mL) was stirred at room temperature for 16 h until the raw materials was all consumed. The reaction mixture was concentrated and purified by column to give A1-6 (12.0 g, yield: 76.9%). MS(ESI)(m/z): 502 ([M+H]⁺).

### Step 8

To the solution of A1-6 (3.0 g, 6.0 mmol) and DIEA (1.1 g) in DMF (15 mL) was add HATU (2.62 g, 6.9 mmol) at −5˜0° C. under N₂ atmosphere. After stirred at 0° C. for 0.5˜1 h, the solution of A1-5 in DMF which was obtained in step 4 was added. The resulting mixture was warm to room temperature and stirred for 2-3 h. After quenched with water (100 mL), the mixture was extracted with EA. The combined organic phase was washed with 1M HCl and brine, dried over anhydrous Na₂SO₄ and filtered. The filtrate was concentrated and purified by column chromatography to give A1-7 (1.6 g, two-step yield: 37.5%). MS(ESI)(m/z): 758 ([M+Na]⁺).

### Step 9

The solution of A1-7 (1.6 g, 2.2 mmol) and DBU (0.3 g, 2 mmol) in DMF (11 mL) was stirred at room temperature for 1.5 h under N₂ atmosphere. After TLC showed A1-7 was all consumed, 10% Pd/C (0.2 g) was added. After stirred at room temperature under H₂ atmosphere for 2 h. The reaction mixture was filtered through celite. The filter cake was washed with the deionized water (20 mL). The filtrate was extracted with DCM (10 mL), The resulting aqueous phase was concentrated to dryness under reduced pressure to give A1-8 (0.9 g, yield: 98.0%).

### Step 10

The solution of A1-8 (0.9 g, 2 mmol), A1-8X (0.5 g, 1.7 mmol) and TEA (0.2 g, 2 mmol) in MeCN (6.3 mL) and water (6.3 mL) was stirred at room temperature under N₂ atmosphere for 2 hours. The reaction mixture was diluted with water (20 mL), extracted with EA (20 mL*2). The aqueous phase was adjusted to pH=4-5 with acetic acid, and then concentrated to dryness under reduced pressure to give A1-9 (1.0 g, yield 95.2%). MS(ESI)(m/z): 639 ([M+Na]⁺).

### Step 11

To the solution of A1-9 (1.0 g, 1.6 mmol) and DMAP (0.26 g) in DCM (16 mL) was added A1-9X (0.68 g, 1.57 mmol) and EDCI (0.4 g). After stirred at room temperature for 16 h, the mixture was washed successively with 10% citric acid aqueous solution, water, sat. Na₂CO₃ aqueous solution, and brine. The organic phase was dried over anhydrous Na₂SO₄ and filtered. The filtrate was concentrated and purified by column chromatography to give A1 (1.2 g, yield: 73.0%). ¹H-NMR (400 MHZ, DMSO-d₆): 10.05 (s, 1H), 9.00 (s, 2H), 8.2 (br s, 1H), 7.85 (s, 2H), 7.44 (m, 1H), 7.10-7.25 (m, 5H), 6.74 (s, 1H), 4.70-4.80 (m, 3H), 4.32 (s, 2H), 4.25 (s, 2H), 4.15 (s, 2H), 4.10-3.55 (m, 15H), 3.40-3.05 (m, 6H), 3.00-1.15 (m, 10H), 0.99 (t, 3H). MS(ESI)(m/z) 1043 ([M+Na]+).

### 2. Preparation of Compound A1-9X

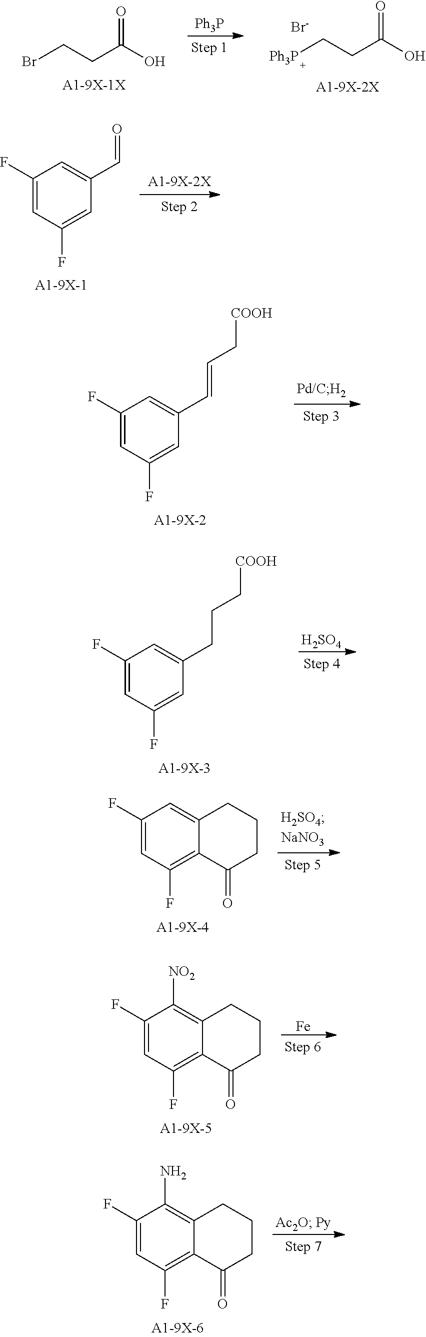

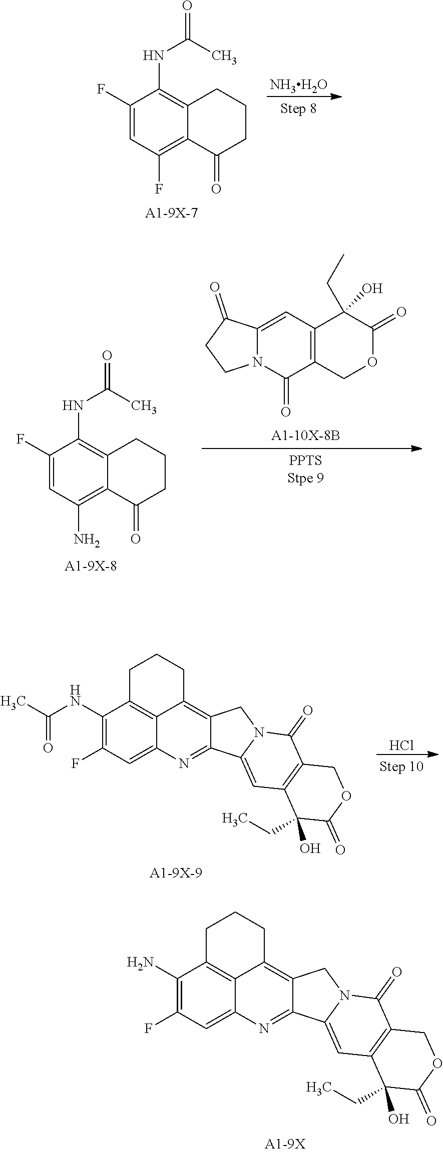

### Step 1

The solution of A1-9X-1X (642 g, 4.2 mol) and triphenylphosphine (1155 g, 4.41 mol) in MeCN (6 L) was refluxed for 24 h. After cooling down, the reaction mixture was concentrated to dryness under reduced pressure. The residua were scattered in MTBE (6 L). The mixture was filtered and the filter cake was washed with MTBE and dried under reduced pressure to give A1-9X-2X (1500 g, yield: 86.0%).

### Step 2

To the solution of A1-9X-1 (426 g, 3 mol) and A1-9X-2X (1.5 kg, 3.6 mol) in THF (2.4 L) was added the solution of t-BuONa (840 g, 7.5 mol) in THF (2.4 L) dropwise at −5° C. under N₂ atmosphere. After stirred at −5° C. for 5-8 h, the reaction mixture was poured into ice water (7.5 L). The resulting mixture was concentrated under reduced pressure to remove most of the THF at 40-45° C., and then extracted with MTBE (5.0 L*3) to remove the by-products. The aqueous phase was adjusted to pH=4-5 with 6.0 N HCl, and then extracted with EA (4.0 L*3). The combined organic phase was washed with water and brine, dried over anhydrous Na₂SO₄ and filtered. The filtrate was concentrated to dryness to give A1-9X-2 (594.0 g, yield: 100%).

### Step 3

To the solution of A1-9X-2 (594.0 g, 3 mol) in ethyl acetate (2.5 L) was added 10% Pd/C (containing 50% water, 75 g). After stirred at 25-35° C. under H₂ atmosphere for 16 h, The reaction mixture was filtered through celite, and the filtrate is concentrated to dryness under reduced pressure to give A1-9X-3 (600.0 g, yield: 100%).

### Step 4

To concentrated sulfuric acid (3 kg) was added A1-9X-3 (600.0 g, 3 mol) slowly to keep the internal temperature below 35° C., After stirred at room temperature for 2-3 h, the reaction mixture was slowly poured into ice-water (6.5 kg). The resulting mixture was extracted with MTBE (3.5 L) and EA (0.5 L). The combined organic phase was washed with sat. NaHCO₃ aqueous solution and brine, dried over anhydrous Na₂SO₄ and filtered. The filtrate was concentrated and purified by column chromatography to give A1-9X-4 (230.0 g, yield: 42.1%). ¹HNMR (400 MHZ, CDCl₃): 6.79-6.69 (m, 2H), 2.96 (t, J=6.0 Hz, 2H), 2.64 (t, J=6.0 Hz, 2H), 2.11 (m, 2H).

### Step 5

To concentrated sulfuric acid (1200 g) was added A1-9X-4 (125 g, 686.8 mmol), while keeping the internal temperature below 5° C., and then sodium nitrate (70 g, 823.5 mmol) was added in batches. After stirred at room temperature for 2-3 h. the reaction mixture was slowly poured into ice water (6.5 kg). The resulting mixture was extracted with MTBE (1.5 L) and EA (150 mL), the combined organic phase was washed with sat. NaHCO₃ aqueous solution and brine, dried over anhydrous Na₂SO₄ and filtered. The filtrate was concentrated and purified by column chromatography to give A1-9X-5 (71.0 g, yield: 45.5%).

### Step 6

To the solution of A1-9X-5 (100 g, 440 mmol) and NH₄Cl (70 g, 1.31 mol) in ethanol (2.1 L) and water (0.3 L) was add iron powder (200 g, 3.57 mol) slowly. After addition, the reaction mixture was heated to 80° C. and stirred for 1-2 h at the same temperature. After cooling down, the reaction mixture was filtered. The filtrate was concentrated under reduced pressure to remove most of ethanol. The residua were diluted with water and then extracted with DCM (2.6 L). The combined organic layer was washed with sat. NaHCO₃ aqueous solution and brine, dried over anhydrous Na₂SO₄ and filtered. The filtrate was directly used in next step as a solution of A1-9X-6 in DCM.

### Step 7

To the solution of A1-9X-6 in DCM which was obtained in step 6 was added pyridine (150 g, 1.9 mol) and DMAP (1.0 g), and then acetic anhydride (99 g, 968 mmol) was added slowly while keeping internal temperature below 15° C. After stirred at room temperature for 2-3 h, the reaction mixture was slowly poured into ice water (1.5 kg). The organic layer was separated, washed with 1.0 N HCl until pH<7, washed with sat. NaHCO₃ aqueous solution and brine, and then concentrated to dryness. The residua were dissolved in methanol (1 L), and then adjusted pH=11-12 with 25.0% NaOH aqueous solution. After stirred at 35-45° C. for 1-3 h, the mixture was concentrated under reduced pressure to remove most of the organic solvent. DCM and water was added and DCM layer was separated and concentrated to dryness. The residua was scattered in MTBE, the solid was collected by filtration and dried under reduced pressure to give A1-9X-7 (55.0 g, yield: 52.3%). ¹H-NMR (400 MHZ, CDCl₃): 6.83 (m, 1H), 2.86 (t, J=6.0 Hz, 2H), 2.64 (t, J=6.0 Hz, 2H), 2.25 (s, 3H), 2.06 (m, 2H).

### Step 8

To the solution of A1-9X-7 (9.0 g, 37.6 mmol) in DMSO (150 mL) was added 25% ammonium hydroxide (250 g) slowly, and then the reaction mixture was stirred at 65-75° C. for 16 h in an autoclave. After cooling down, the reaction mixture was diluted with an appropriate amount of water, and then extracted with ethyl acetate multiple times. The combine organic layer was washed with water and brine, dried over anhydrous Na₂SO₄ and filtered. The filtrate was concentrated and purified by column chromatography to give A1-9X-8 (4.5 g, yield: 50.6%). ¹H-NMR (400 MHZ, DMSO-d₆): 9.09 (s, 1H), 6.43 (d, J=12.4 Hz, 1H), 2.66 (t, J=6.0 Hz, 2H), 2.50 (m, 2H), 2.00 (s, 3H), 1.87 (t, J=6.0 Hz, 2H). LC-MS: (m/z): 237.1 [M+H]⁺.

### Step 9

The solution of A1-9X-8 (4 g, 17 mmol), A1-10X-8B (4.9 g, 18.6 mmol) and PPTS (4.8 g, 17 mmol) in toluene (800 mL) was stirred at 120° C. under N₂ atmosphere for 8 hours. After cooling down, the reaction mixture was concentrated under reduced pressure to dryness. The residua was scattered in acetone (50 mL), after cooled down to 0-5° C., the solid was obtained by filtration and dried under reduced pressure to give A1-9X-9 (6.5 g, yield: 82.5%). LC-MS: (m/z): 464.2 [M+H]⁺.

### Step 10

The mixture of A1-9X-9 (5.0 g, 10.8 mmol), concentrated HCl (25 mL) and water (25 mL) was stirred at 80° C. for 1 h, and then concentrated to dryness under reduced pressure. The residua was scattered in acetone (30 mL), and the solid was collected by filtration and dried under reduced pressure to give A1-9X (3.1 g, yield: 68.1%). LC-MS: (m/z): 422.1 [M+H]⁺.

### 3. Preparation of Compound A2˜A6

Following the general procedures of compound A1, replaced A1-3X with:

| | |
|---|---|
| 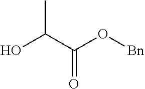 | A2-3X |
| 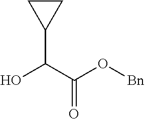 [SMILES: O=C(OCc1ccccc1)C(O)C1CC1] | A3-3X |
| 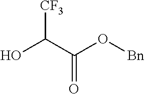 [SMILES: O=C(OCc1ccccc1)C(O)C(F)(F)F] | A4-3X |
| 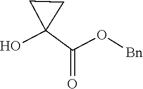 [SMILES: O=C(OCc1ccccc1)C1(O)CC1] | A5-3X |
| 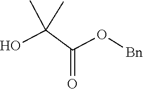 | A6-3X |

### 4. Preparation of Compound A7 and A8

Following the general procedures of compound A1 and A5, replaced A1-9X with A1-10X:

| | |
|---|---|
| A1-10X | 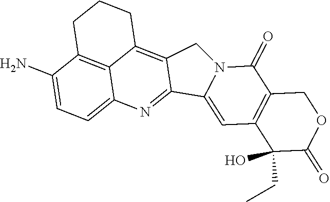 [SMILES: CC[C@@]1(O)C(=O)OCc2c1cc1n(c2=O)Cc2c-1nc1ccc(N)c3c1c2CCC3] |

### 5. Preparation of Compound A1-10X

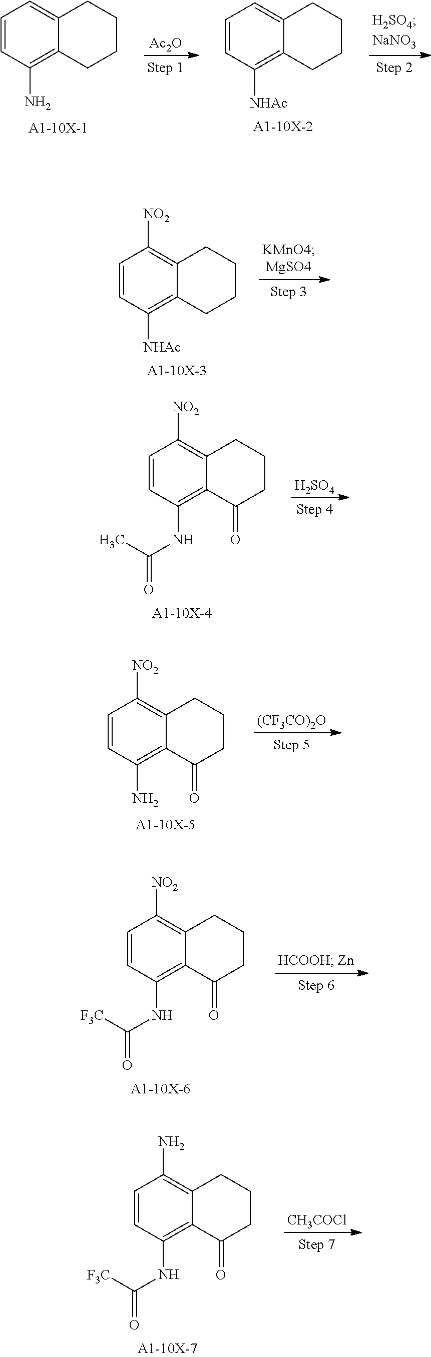

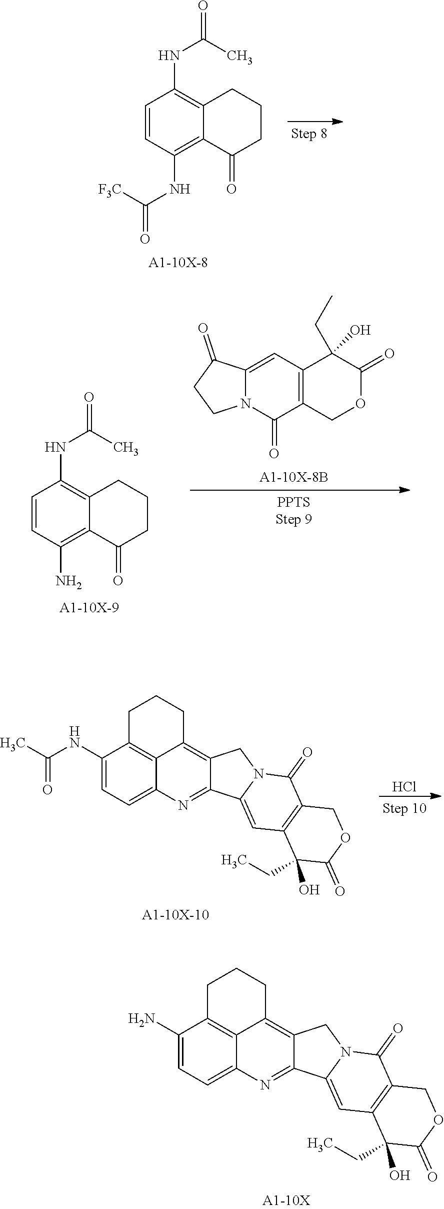 [SMILES: CC(=O)Nc1ccc(NC(=O)C(F)(F)F)c2c1CCCC2=O ; CC(=O)Nc1ccc(N)c2c1CCCC2=O ; CC[C@@]1(O)C(=O)OCc2c1cc1n(c2=O)CCC1=O ; CC[C@@]1(O)C(=O)OCc2c1cc1n(c2=O)Cc2c-1nc1ccc(NC(C)=O)c3c1c2CCC3 ; Cl ; CC[C@@]1(O)C(=O)OCc2c1cc1n(c2=O)Cc2c-1nc1ccc(N)c3c1c2CCC3]

### Step 1

To the solution of A1-10X-1 (914 g, 6.22 mol) and TEA (754 g, 7.46 mol) in DCM (6 L) was dropped acetic anhydride (698 g, 6.84 mol) at 0° C. over 1 h. After stirred for another 30 min, the reaction mixture was quenched with water (1 L). The DCM layer was separated and the aqueous layer was extracted with DCM (800 mL). The combines organic phase was adjust pH ˜ 2 with 1N HCl. The organic phase was separated, and then the aqueous layer was extracted with DCM once. The combined organic phase was washed with sat. NaHCO₃ aqueous solution (1 L), dried over Na₂SO₄ and filtered. The filtrate was concentrated to dryness under reduced pressure to give A1-10X-2 (1150 g, yield: 97.8%).

### Step 2

To the solution of A1-10X-2 (300 g, 1.58 mol) in sulfuric acid (2 L) was added sodium nitrate (134.3 g, 1.58 mol) in batches within 2 hours at 0° C. After stirred for 30 minutes, the reaction mixture was slowly poured into ice water (8 L), and then filtered. The filter cake was washed twice with ice water and then dissolved in DCM (1 L). The resulting solution was washed with sat. NaHCO₃ until PH>7, and then dried over Na₂SO₄ and filtered. The filtrate was concentrated to dryness under reduced pressure. The residua was scattered in MTBE (400 mL), after cooled down to 0-5° C., the solid was obtained by filtration and dried under reduced pressure to give A1-10X-3 (264 g, yield: 71.3%).

### Step 3

To the solution of A1-10X-3 (160 g, 680 mmol) in acetone (5 L) was added a solution of MgSO₄ (111.6 g, 930 mmol) in water (620 mL) and then KMnO₄ (323 g, 2.05 mol) was added in batches over 3 h at 0° C. After stirred at room temperature for 1.5 h, the reaction mixture was quenched with 40% sodium thiosulfate aqueous solution (2 L) until no oxidation was detected and then filtered. The filtrate was concentrated to most of acetone, and then extracted with DCM (6 L). The organic layer was washed with sat. NaHCO₃ aqueous solution and brine, dried over anhydrous Na₂SO₄ and filtered. The filtrate was concentrated and purified by column chromatography to give A1-10X-4 (100 g, yield: 59.2%). LC-MS: (m/z): 274.3 [M+Na]⁺.

### Step 4

The mixture of A1-10X-4 (80 g, 320 mmol) and concentrated HCl (500 mL), was stirred at 90-100° C. for 2 h. After cooled down to 0-10° C., the reaction mixture was poured into ice water (2 L). Filtered, the cake was rinsed with ice water and dried under reduced pressure to give A1-10X-5 (66 g, yield: 100%).

### Step 5

To the solution of A1-10X-5 (66 g, 320 mmol) and pyridine (52 mL, 650 mmol) in DCM (1.5 L) was added trifluoroacetic anhydride (94 mL, 676 mmol) dropwise within 40 min at 0° C. After stirred for another 20 min, the reaction mixture was concentrated to dryness under reduced pressure. The residua was dissolved in DCM (1 L), washed with 1N HCl to remove pyridine, and then washed with sat. NaHCO₃ aqueous solution and brine. The organic phase was dried and concentrated to dryness under reduced pressure to give A1-10X-6 (62 g, yield: 64.1%).

### Step 6

To the solution of A1-10X-6 (54 g, 180 mmol) in DCM (100 mL), methanol (1 L), formic acid (50 mL) and water (50 mL) was added zinc powder (150 g, 2.29 mol) in batches within 50 min at 0° C. After stirred for another 1.5 h, the reaction mixture was filtered, the organic layer was separated and washed with sat. NaHCO₃ aqueous solution and brine. The organic phase was dried and concentrated to dryness under reduced pressure to give A1-10X-7 (42 g, yield: 85.7%).

### Step 7

To the solution of A1-10X-7 (40 g, 147.0 mmol) and TEA (49 mL, 352.5 mmol) in DCM (1.6 L) was added acetyl chloride (27 mL, 380.0 mmol) dropwise at 0-5° C. After stirred for another 150 min, the reaction mixture was quenched with water (600 mL). Filtered and the filter cake was scattered in water. The solid was collected by filtration and dried under reduced pressure to give A1-10X-8 (46.2 g, yield: 100%). 1H-NMR (400 MHZ, DMSO-d₆): 13.30 (s, 1H), 9.60 (s, 1H), 8.31 (d, J=9.2 Hz, 1H), 7.72 (d, J=9.2 Hz, 1H), 2.86 (t, J=7.0 Hz, 1H), 2.74 (t, J=1.6 Hz, 1H), 2.00 (m, 1H). LC-MS: (m/z): 315.1 [M+H]⁺.

### Step 8

To the solution of A1-10X-8 (30 g, 96 mmol) in methanol (2 L) and water (150 mL) was added K₂CO₃ (50 g, 360 mmol) at 50° C. After stirred for another 20 min. The reaction mixture was cooled to room temperature and then concentrated to dryness under reduced pressure. The residua was scattered in water (400 mL). Filtered, the cake was scattered in water (300 mL). The solid was collected by filtration and dried under reduced pressure to give A1-10X-9 (7.8 g, yield: 37.3%). LC-MS: (m/z): 219.1 [M+H]⁺.

### Step 9

The mixture of A1-10X-9 (6 g, 27.5 mmol), A1-10X-8B (8 g, 30.3 mmol) and PPTS (6.9 g, 27.5 mmol) in toluene (1 L) was stirred at 120° C. for 4 h under N₂ atmosphere. After cooled to 0-5° C., the reaction mixture was filtered. The filter cake was dissolved in DCM and methanol, and concentrated to dryness under reduced pressure. The residua was scattered in acetone (50 mL) at 0-5° C., the solid was obtained by filtration and dried under reduced pressure to give A1-10X-10 (12.2 g, yield: 100%). LC-MS: (m/z): 446.2 [M+H]⁺.

### Step 10

The mixture of A1-10X-10 (12.2 g, 27.5 mmol) in concentrated HCl (80 mL) and water (80 mL) was stirred at 80° C. for 2.5 h. After cooled to room temperature, the reaction mixture was diluted with a small amount of methanol, and then concentrated to dryness under reduced pressure. The residua was scattered in acetone (30 mL) at room temperature. The solid was obtained by filtration and dried under reduced pressure to give A1-10X (11.1 g, yield: 100%). ¹H-NMR (400 MHZ, DMSO-d₆): 8.28 (d, J=7.2 Hz, 1H), 7.45 (br s, 2H), 7.25 (d, J=7.2 Hz, 1H), 6.70 (s, 1H), 4.65 (m, 2H), 4.20 (s, 2H), 3.50 (m, 2H), 2.73 (m, 2H), 2.20 (m, 2H), 1.81 (m, 1H), 0.91 (t, J=5.0 Hz, 2H). LC-MS: (m/z): 404.1 [M+H]⁺. LC-MS: (m/z): 404.1 [M+H]⁺.

### 6. Preparation of Compound A10

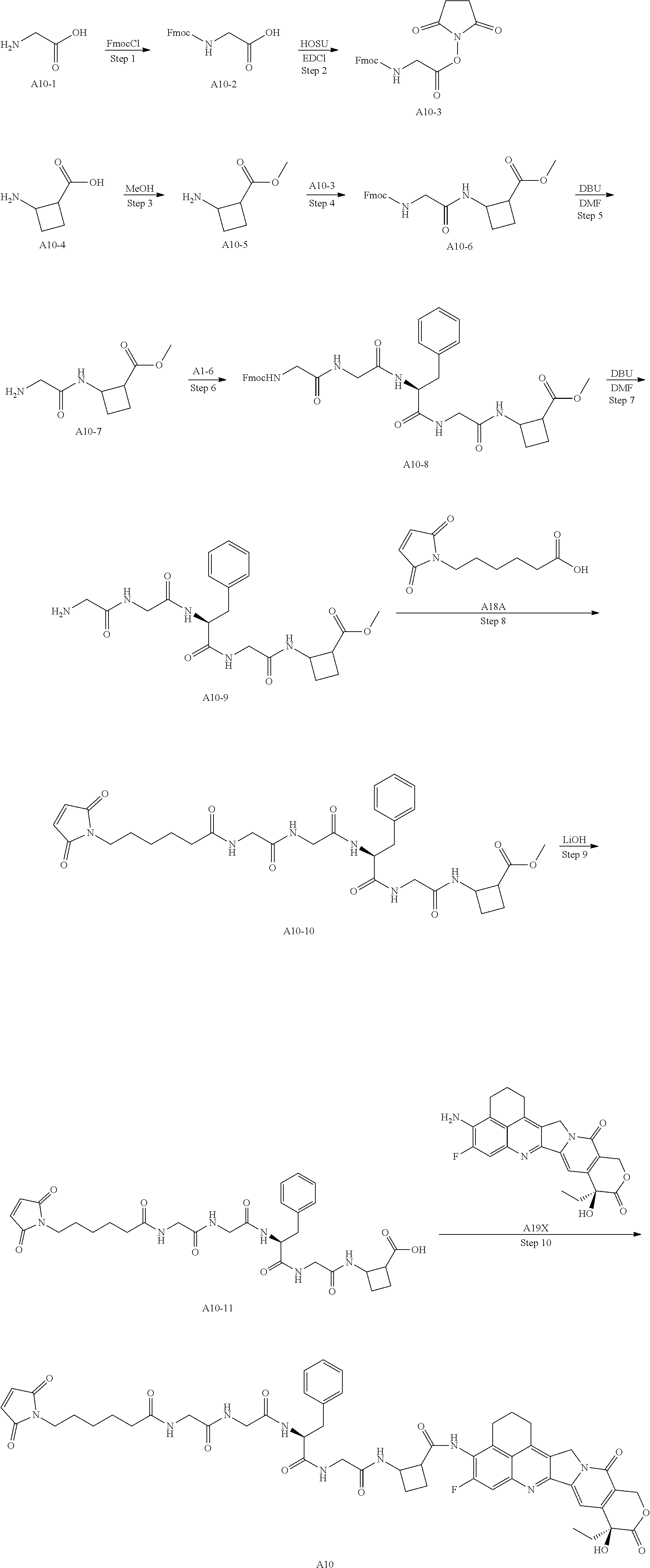

### Step 1

To the solution of A10-1 (5.0 g, 66.6 mmol) and Fmoc-Cl (20.6 g, 79.6 mmol) in 1,4-dioxane (50 mL) was added 1M Na₂CO₃ aqueous solution (18 mL) dropwise at 0° C. (internal temperature below 5° C.). After the addition was completed, the reaction mixture was stirred at room temperature for 2 h. Then adjust pH ˜2 with 1M HCl (60 mL) at 0˜5° C. The resulting mixture was extracted with ethyl acetate (100 mL*3). The combined organic phase was concentrated under reduced pressure to give 15 g crude product. The crude product was scattered in MTBE (50 mL), and stirred at room temperature for 30 min. The solid was collected by filtration and dried under reduced pressure to give A10-2 (17.9 g, yield: 90.3%). MS(ESI)(m/z): 298 ([M+H]+).

### Step 2

The suspension of A10-2 (17.9 g, 60.2 mmol), HOSU (7.7 g, 66.9 mmol), and EDCI (13.8 g, 72.0 mmol) in MeCN (180 mL) was stirred at room temperature for 16 h. After removing MeCN under reduced pressure, the residua was diluted with sat. NaHCO₃ aqueous solution (270 mL), and extracted with DCM. The combined organic phase was concentrated and purified by silica gel column to give A10-3 (19.2 g, yield: 84.3%). MS(ESI)(m/z): 401 ([M+Na]⁺).

### Step 3

To the solution of A10-4 (5.0 g, 43.4 mmol) in methanol (75 mL) was added thionyl chloride (10.3 g, 86.6 mmol) dropwise at −5˜0° C. After stirred at room temperature for 1 h, the reaction mixture was heated to reflux for 1 hour. Cooled down, the reaction mixture was concentrated and purified by column chromatography to give A10-5 (5.16 g, yield: 92.0%).

### Step 4

The mixture of A10-5 (5.0 g, 38.7 mmol), A10-3 (17.6 g, 46.5 mmol) and triethylamine (8.2 g, 81.0 mmol) in MeCN (50 mL) and water (50 mL) was stirred at room temperature under N₂ atmosphere for 2 h. The reaction mixture was quenched with water (80 mL), and extracted with ethyl acetate (50 mL*2). The aqueous layer was adjusted to pH=4-5 with acetic acid, and then concentrated to dryness under reduced pressure to give A10-6 (11.9 g, yield: 75.3%). MS(ESI)(m/z): 431 ([M+Na]⁺).

### Step 5

The solution of A10-6 (11 g, 26.9 mmol) and DBU (4.1 g, 26.9 mmol) in DMF (55 mL) was stirred at room temperature for 3-4 h under N₂ atmosphere. The reaction mixture was directly used in next step without further purification as a solution of A10-7 in DMF.

### Step 6

To the solution of A1-6 (13.5 g, 26.9 mmol) and DIEA (4.9 g, 37.9 mmol) in DMF (55 mL) was added HATU (11.8 g, 31.0 mmol) at −5˜0° C. under N₂ atmosphere. After stirred at 0° C. for 0.5˜1 h, the above solution of A10-7 in DMF was added. The resulting mixture was warm to room temperature and stirred for 2-3 h. After quenched with water (200 mL), the mixture was extracted with EA. The combined organic phase was washed with 1M HCl and brine, dried over anhydrous Na₂SO₄ and filtered. The filtrate was concentrated and purified by column chromatography to give A10-8 (8.1 g, yield for 2 steps: 45.0%). MS(ESI)(m/z): 693 ([M+Na]⁺).

### Step 7

The solution of A10-8 (8.0 g, 11.9 mmol) and DBU (1.8 g, 11.8 mmol) in DMF (40 mL) was stirred at room temperature for 3-4 h under N₂ atmosphere. The reaction mixture was directly used in next step without further purification as a solution of A10-9 in DMF.

### Step 8

To the solution of A1-8A (2.5 g, 11.8 mmol) and DIEA (2.2 g, 17.0 mmol) in DMF (12.5 mL) was add HATU (5.2 g, 13.7 mmol) at −5˜0° C. under N₂ atmosphere. After stirred at 0° C. for 0.5˜1 h, the above solution of A10-9 in DMF was added. The resulting mixture was warm to room temperature and stirred for 2-3 h. After quenched with water (200 mL), the mixture was extracted with EA. The combined organic phase was washed with 1M HCl and brine, dried over anhydrous Na₂SO₄ and filtered. The filtrate was concentrated and purified by column chromatography to give A10-10 (4.78 g, yield for 2 steps: 62.8%). MS(ESI)(m/z): 663 ([M+Na]⁺).

### Step 9

To the solution of A10-10 (4.7 g, 7.3 mmol) in dry methanol (50 mL) was added LiOH (0.63 g, 26.3 mmol). After stirred at room temperature for 2 h, the reaction mixture was adjusted to pH=4˜5 with acetic acid. The resulting mixture was concentrated to dryness under reduced pressure to give A10-11 (4.1 g, yield: 89.1%).

### Step 10

To the solution of A10-11 (4.0 g, 6.4 mmol) and DMAP (1.1 g, 9.0 mmol) in DCM (50 mL) was added A1-9X (2.7 g, 6.4 mmol) and EDCI (1.5 g). After stirred at room temperature for 16 h, the reaction mixture was washed successively with 10% citric acid aqueous solution, water, sat. Na₂CO₃ aqueous solution, and brine. The organic phase was dried over anhydrous Na₂SO₄ and filtered. The filtrate was concentrated and purified by column chromatography to give A10 (4.0 g, yield: 60.6%). ¹H-NMR (400 MHZ, DMSO-d₆): δ 10.05 (s, 1H), 9.00 (s, 2H), 8.2 (br s, 1H), 7.85 (s, 2H), 7.44 (m, 1H), 7.10-7.25 (m, 5H), 6.74 (s, 1H), 4.70-4.80 (m, 3H), 4.32 (s, 2H), 4.25 (s, 2H), 4.15 (s, 2H), 4.10-3.55 (m, 15H), 3.40-3.05 (m, 6H), 3.00-1.15 (m, 10H), 0.99 (t, 3H). MS(ESI)(m/z): 1053 ([M+Na]⁺).

### 7. Preparation of Compound A9, A11, A12

Following the general procedures of compound A10, replaced A10-4 with:

| | |
|---|---|
| A9-4 | 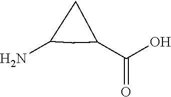 |
| A11-4 | 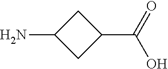 |
| A12-4 | 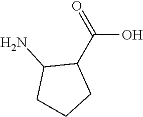 |

### 8. Preparation of Compound A13

Following the general procedures of compound A11, replaced A1-9X with A1-10X:

| | |
|---|---|
| A1-10X | 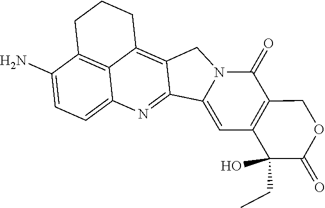 [SMILES: CC[C@@]1(O)C(=O)OCc2c1cc1n(c2=O)Cc2c-1nc1ccc(N)c3c1c2CCC3] |

### 9. Preparation of Compound A15

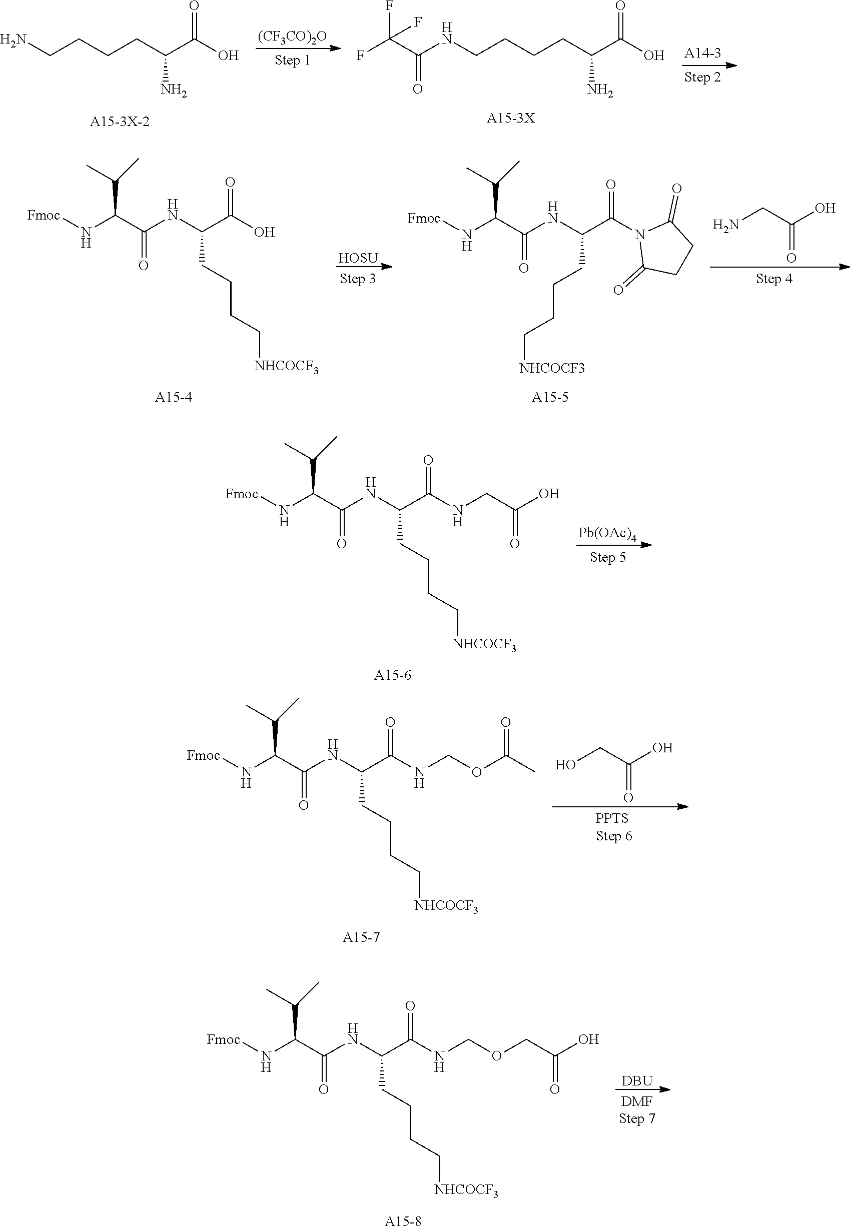

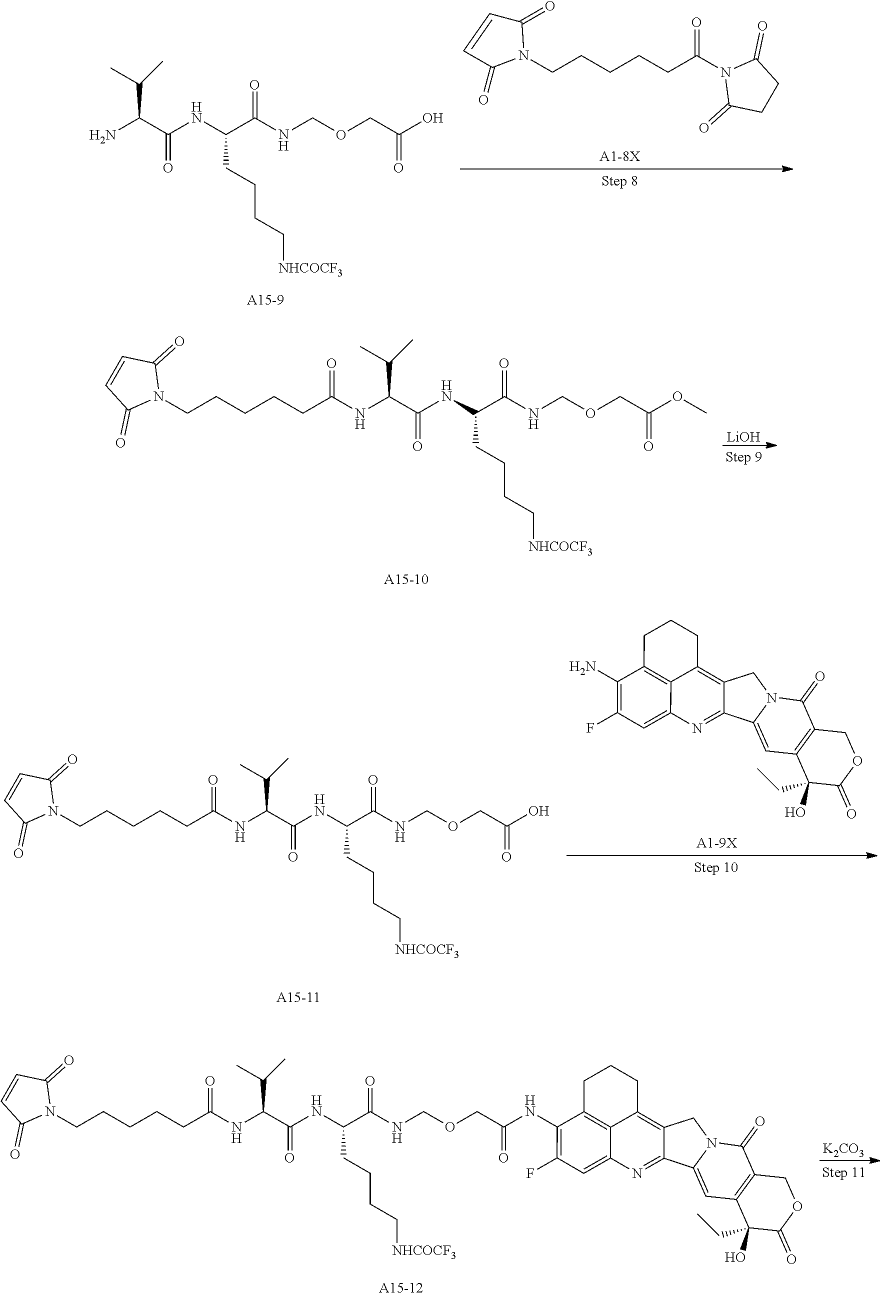

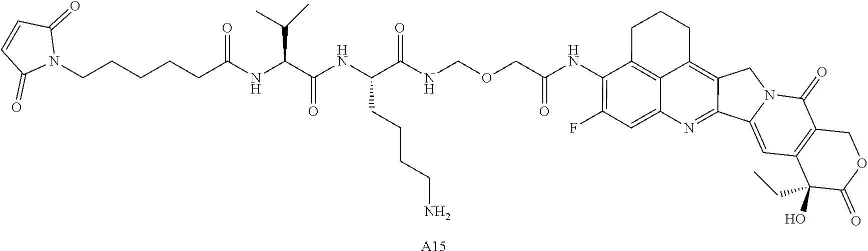

### Step 1

To the solution of A15-3X-2 (50 g, 342 mmol) and pyridine (13.5 g, 171 mmol) in DCM (500 mL) was added trifluoroacetic anhydride (35.9 g, 171 mmol) dropwise at 0° C. within 40 min. After stirred for another 20 min, the reaction mixture was concentrated to dryness and the residua was redissolved in DCM (1 L). The resulting mixture was washed with 1N HCl to remove pyridine, then washed with sat. Na₂CO₃ aqueous solution and brine, dried over anhydrous Na₂SO₄ and filtered. The filtrate was concentrated and purified by column chromatography to give A15-3X (13.2 g, yield: 32.0%).

### Step 2

The solution of A15-3X (10 g, 41.3 mmol), A14-3 (17.4 g, 41.3 mmol) and TEA (41.8 g, 41.3 mmol) in MeCN (100 mL) and H₂O (100 mL) was stirred at room temperature for 2 h. The reaction mixture was diluted with water (200 mL) and extracted with EA (200 mL*2). The aqueous phase was adjusted pH=4˜5 with acetic acid, and then concentrated to dryness under reduced pressure to give A15-4 (17.7 g, yield: 76.3%).

### Step 3

The suspension of A15-4 (17.7 g, 31.4 mmol), HOSU (4.0 g, 34.5 mmol), and EDCI (7.2 g, 37.7 mmol) in MeCN (180 mL) was stirred at room temperature for 16 h. After removing MeCN under reduced pressure, the residua was diluted with the of sat. NaHCO₃ aqueous solution (100 mL), and extracted with DCM. The combined organic phase was concentrated and purified by silica gel column chromatography to give A15-5 (16.4 g, yield: 81.3%).

### Step 4

The solution of Glycine (1.9 g, 24.8 mmol), A15-5 (16.0 g, 24.8 mmol) and TEA (2.5 g, 24.8 mmol) in MeCN (50 mL) and H₂O (50 mL) was stirred at room temperature under N₂ atmosphere for 1.5 h. The reaction mixture was diluted with water (80 mL) and extracted with EA (50 mL*2). The aqueous phase was adjusted pH=4˜5 by acetic acid, and then concentrated to dryness under reduced pressure to give A15-6 (13.2 g, yield: 85.6%).

### Step 5

To the solution of A15-6 (13.0 g, 20.9 mmol) and acetic acid (55 mL) in THF (90 mL) was added Pb(OAc)₄ (37.1 g, 83.6 mmol) at 40° C. under N₂ atmosphere, and then the resulting mixture was heated to reflux and kept for 16 h. After chilling to room temperature, the reaction mixture was filtered through celite, washed with EA, the filtrate was concentrated and purified by silica gel column to give A15-7 (13.2 g, yield: 100%).

### Step 6

The mixture of A15-7 (13.0 g, 20.5 mmol), hydroxyacetic acid (6.2 g, 82 mmol) and PPTS (1.0 g, 4.1 mmol) in DCM (100 mL) was refluxed at 45° C. under N₂ atmosphere for 2 h. The reaction solution was concentrated under reduced pressure and purified by column chromatography to give A15-8 (6.8 g, yield: 51.1%).

### Step 7

The solution of A15-8 (6.5 g, 10.0 mmol) and DBU (1.5 g, 10.0 mmol) in DMF (32.5 mL) was stirred at room temperature for 3-4 h under N₂ atmosphere. The reaction mixture was directly used in next step without further purification as a solution of A15-9 in DMF.

### Step 8

To the solution of A1-8X (2.9 g, 10.0 mmol) and DIEA (1.8 g, 14.0 mmol) in DMF (45 mL) was add HATU (4.4 g, 11.5 mmol) at −5˜0° C. under N₂ atmosphere. After stirred at 0° C. for 0.5˜1 h, the solution of A15-9 in DMF which was obtained in step 7 was added. The resulting mixture was warm to room temperature and stirred for 2-3 h. After quenched with water (100 mL), the mixture was extracted with EA. The combined organic phase was washed with 1M HCl and brine, dried over anhydrous Na₂SO₄ and filtered. The filtrate was concentrated and purified by column chromatography to give A15-10 (3.1 g, yield for 2 steps: 49.2%).

### Step 9

To the solution of A15-10 (3.0 g, 4.7 mmol) in dry methanol (20 mL) was added LiOH (0.45 g, 18.8 mmol). After stirred at room temperature for 3 h, the reaction mixture was adjusted pH=4˜5 with acetic acid. The mixture was concentrated to dryness under reduced pressure to give A15-11 (2.7 g, yield: 93.4%).

### Step 10

To the solution of A15-11 (2.5 g, 4.0 mmol), and DMAP (0.67 g, 5.5 mmol) in DCM (20 mL), was added A1-9X (1.7 g, 4.0 mmol) and EDCI (0.95 g, 5.0 mmol). After stirred at room temperature for 16 h, the reaction mixture was washed successively with 10% citric acid aqueous solution, water, sat. Na₂CO₃ aqueous solution, and brine. The organic phase was dried over anhydrous Na₂SO₄ and filtered. The filtrate was concentrated and purified by column chromatography to give A15-12 (2.8 g, yield: 68.7%)

### Step 11

To the solution of A15-12 (2.5 g, 2.4 mmol) in methanol (25 mL) and water (12.5 mL) was added K₂CO₃ (1.3 g, 9.6 mmol) at 50° C. After stirred for another 20 min, the reaction mixture was cooled to room temperature and concentrated to dryness under reduced pressure. The residua was scattered in water (40 mL). Filtered, the cake was scattered in water (30 mL). The solid was collected by filtration and dried under reduced pressure to give A15 (0.83 g, yield: 37.3%).

### 10. Preparation of Compound A14

Following the general procedures of compound A15, replaced A15-3X with D-alanine:

| | |
|---|---|
| D-alanine | 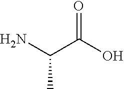 |

### 11. Preparation of Compound A16

Following the general procedures of compound A15, replaced A15-3X with D-alanine, replaced A1-9X with A1-10X:

| | |
|---|---|
| D-alanine | 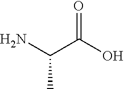 |

| | |
|---|---|
| A1-10X | 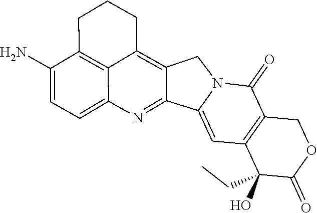 [SMILES: CC[C@@]1(O)C(=O)OCc2c1cc1n(c2=O)Cc2c-1nc1ccc(N)c3c1c2CCC3] |

### 12. Preparation of Compound A17

Following the general procedures of compound A1, replaced 6-aminocaproic acid with 3-(2-(2-(2-aminoethoxy) ethoxy) ethoxy) propanoic acid:

| | |
|---|---|
| 3-(2-(2-(2- aminoethoxy)ethoxy)ethoxy)propanoic acid | 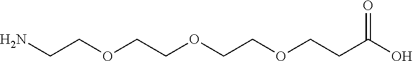 [SMILES: NCCOCCOCCOCCC(=O)O] |

### 13. Preparation of Compound A18

Following the general procedures of compound A15, replaced A15-3× with D-alanine, replaced A1-9X with A1-10X:
replaced 6-aminocaproic acid with 3-(2-(2-(2-aminoethoxy)ethoxy)ethoxy) propanoic acid:

| | |
|---|---|
| D-alanine | 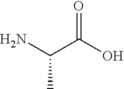 |
| A1-10X | 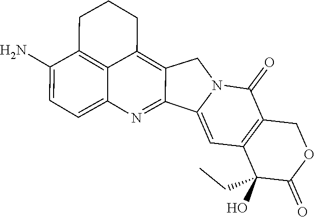 [SMILES: CC[C@@]1(O)C(=O)OCc2c1cc1n(c2=O)Cc2c-1nc1ccc(N)c3c1c2CCC3] |
| 3-(2-(2-(2- aminoethoxy)ethoxy)ethoxy)propanoic acid | 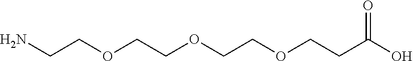 [SMILES: NCCOCCOCCOCCC(=O)O] |

### 14. Preparation of Compound A20

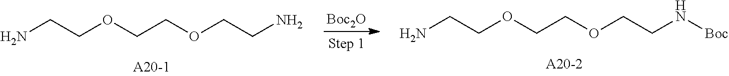 [SMILES: "Boc2O" ; NCCOCCOCCN ; CC(C)(C)OC(=O)NCCOCCOCCN]

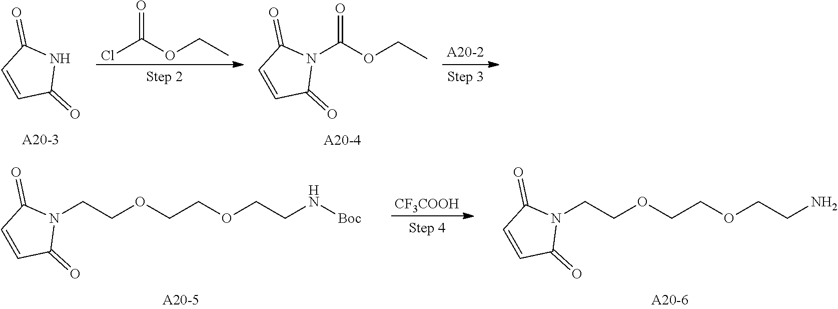

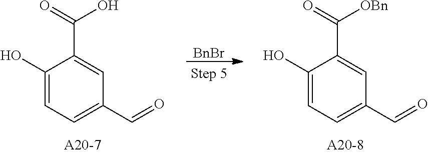

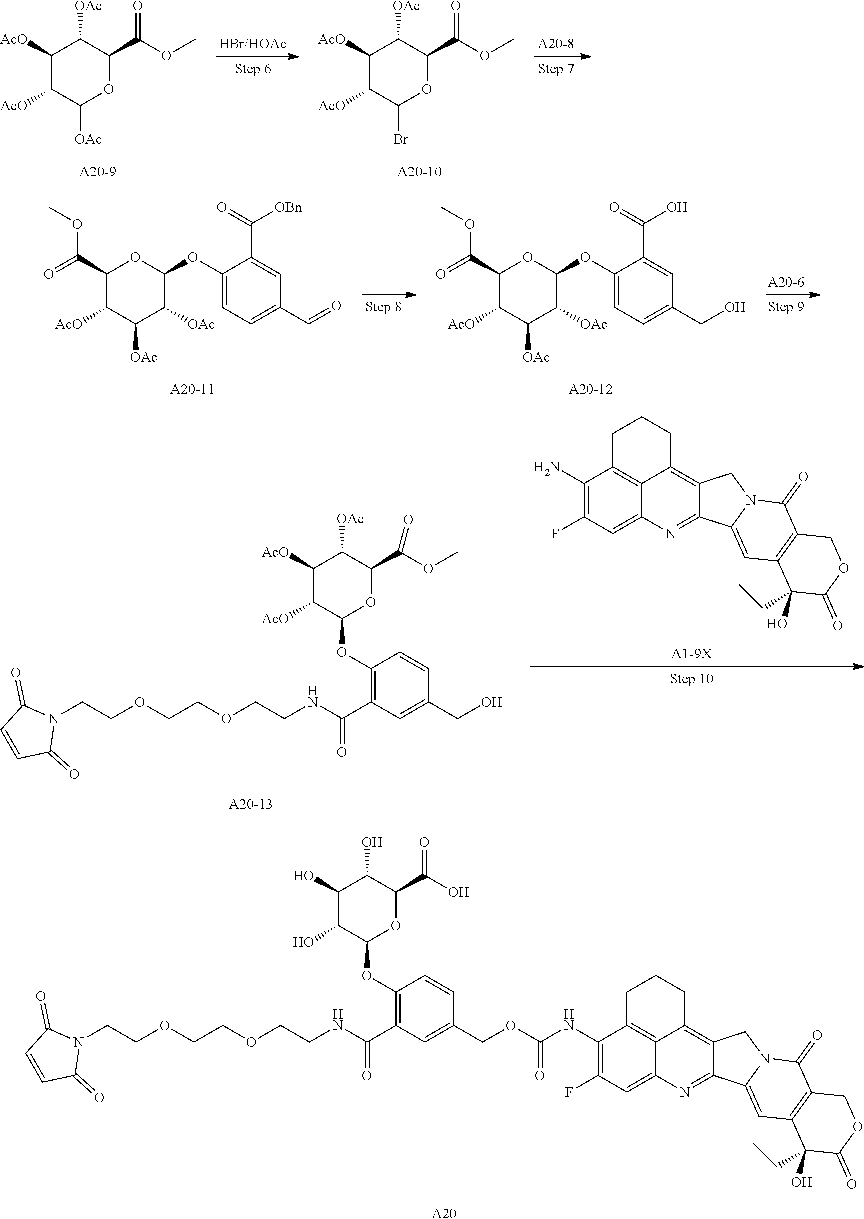

### Step 1

To the solution of A20-1 (10.0 g, 67.5 mmol) in DCM (500 mL) was added Boc₂O (7.4 g, 33.9 mmol) dropwise at 0-5° C. and then stirred for 5 h at the same temperature until (Boc) 20 was all consumed. The reaction mixture was washed with water (200 mL), the aqueous layer was extracted with DCM (50 mL), the combined organic phase was dried and concentrated to dryness under reduced pressure to give A20-2 (8.4 g, yield: 100%).

### Step 2

To the solution of A20-3 (9.7 g, 100 mmol) and N-methylmorpholine (11 mL, 100 mmol) in ethyl acetate (50 mL) was added ethyl chloroformate (7.7 mL, 80.5 mmol) slowly at 0-5° C. After stirred for another 1 h at room temperature, the reaction mixture was quenched with water (50 mL). The aqueous layer was separated and extracted with ethyl acetate (30 mL*2). The combined organic phase was concentrated and purified by column chromatography to give A20-4 (7.3 g, yield: 54.0%).

### Step 3

The mixture of A20-2 (3.0 g, 12.1 mmol) in sat. NaHCO₃ aqueous solution (90 mL) was stirred at room temperature for 15 min and then filtered. To the filtrate was added A20-4 (2.0 g, 11.8 mmol) at 0˜5° C. After stirred at room temperature for 16 h, the reaction mixture was concentrated to dryness under reduced pressure and then remove water through azeotropic distillation with toluene. To the residua was added sodium acetate (10 g, 121.9 mmol) and acetic anhydride (20 mL, 220 mmol). After stirred at 120° C. for 0.5 h, the resulting mixture was cooled down and concentrated to dryness under reduced pressure. The residua was diluted with water (50 mL) and extracted with DCM (50 mL*2). The combined organic phase was dried over anhydrous Na₂SO₄ and concentrated to dryness under reduced pressure to give A20-5 (5.7 g, yield: 100%).

### Step 4

To the solution of A20-5 (5.7 g, 17.4 mmol) in DCM (50 mL) was added TFA (10 g, 87.7 mmol). After stirring at room temperature for 2-3 h, the reaction mixture precipitated. The solid was collected by filtration and dried under reduced pressure to give A20-6 (5.3 g, yield: 89.1%).

### Step 5

To the mixture of A20-7 (15.0 g, 90.3 mmol) and K₂CO₃ (9.1 g, 90.9 mmol) in DMF (240 mL) was added benzyl bromide (10.8 mL, 90.9 mmol) dropwise at 0-5° C. under N₂ atmosphere. After stirred at room temperature for 16 h, the reaction mixture was poured into ice water and stirred for 1 hour. Filtered, the filter cake was dissolved in ethyl acetate (50 mL), dried over anhydrous Na₂SO₄ and filtered. The filtrate was concentrated to dryness under reduced pressure to give A20-8 (20.4 g, yield: 88.3%).

### Step 6

To 40% hydrogen bromide/acetic acid solution (25 mL) was slowly added A20-9 (5.0 g, 13.3 mmol) in batches at 0-5° C. After stirred at room temperature for 2 h, the reaction mixture was diluted with toluene (50 mL) and then concentrated to dryness under reduced pressure. The residua was diluted with ethyl acetate (100 mL), washed with saturated NaHCO₃ aqueous solution (100 mL). The organic phase was dried over anhydrous Na₂SO₄ and filtered. The filtrate was concentrated to dryness under reduced pressure to give A20-10 (5.1 g, yield: 96.6%).

### Step 7

The mixture of A20-8 (2.69 g, 10.5 mmol), A20-10 (5.0 g, 12.6 mmol), activated molecular sieve (5.0 g) and silver oxide (9.3 g, 40.1 mmol) in MeCN (50 mL) were stirred at room temperature under N₂ atmosphere for 2 h. TLC (PE/EA=10/1, UV) showed the raw materials was all consumed. The reaction mixture was diluted with water (100 mL) and then filtered. The filtrate was extracted with ethyl acetate (150 mL*3). The combined organic phase was dried over anhydrous Na₂SO₄ and filtered. The filtrate was concentrated to dryness under reduced pressure to give A20-11 (6.0 g, yield: 100%). MS(ESI)(m/z): 595 ([M+Na]⁺).

### Step 8

To the mixture of A20-11 (6.0 g, 10.5 mmol) and 200-300 mesh silica gel (6.0 g) in isopropanol (18 mL) and DCM (90 mL) was slowly added NaBH₄ (0.8 g, 21.1 mmol) in batches at 0-5° C. After stirred at room temperature for 2 h, the reaction mixture was quenched with water (100 mL), and then extracted with ethyl acetate. The combined organic phase was dried over anhydrous Na₂SO₄ and filtered. The filtrate was concentrated to dryness under reduced pressure. To the solution of the residua in ethanol (275 mL) was added 10% Pd/C (0.55 g). The resulting mixture was stirred at room temperature under H₂ atmosphere for 10-20 min and then filtered through celite. The filter cake was rinsed with ethanol, and the filtrate was concentrated to dryness under reduced pressure to give A20-12 (4.2 g, yield for 2 steps: 81.9%).

### Step 9

To the solution of A20-12 (2.8 g, 5.8 mmol) in DMF (20 mL) was added TEA (1.7 g, 16.8 mmol), 4-(4,6-dimethoxytriazin-2-yl)-4-methylmorpholine hydrochloride (3.5 g, 11.9 mmol) and A20-6 (2.0 g, 5.8 mmol) at 0-5° C. After stirred at room temperature for 1 hour, the reaction mixture was quenched with water (50 mL), and extracted with DCM (50 mL*3). The combined organic phase was dried over anhydrous Na₂SO₄ and filtered. The filtrate was concentrated to dryness under reduced pressure and purified by column chromatography to give A20-13 (1.4 g, yield: 32.2%).

### Step 10

To the solution of A1-9X (0.7 g, 1.7 mmol) and DMAP (0.26 g, 2.1 mmol) in MeCN (20 mL) was added triphosgene (0.5 g, 1.7 mmol) at room temperature and then stirred for 2 h. After A20-13 (1.2 g, 1.7 mmol) was added, the resulting mixture was stirred at room temperature for another 16 h. Then the reaction mixture was concentrated and purified by column chromatography to given A20 (1.15 g, yield: 68.8%).

### 15. Preparation of Compound A19-4X

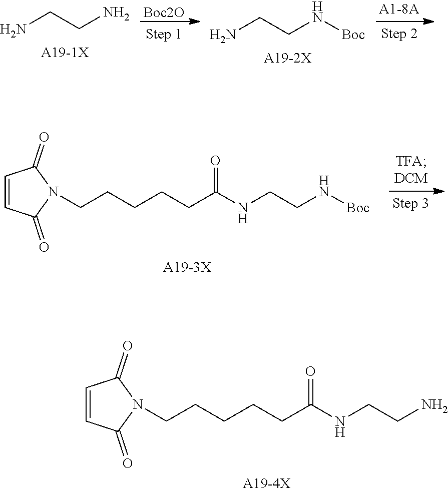

### Step 1

To the solution of A19-1X (10.0 g, 166.4 mmol) in DCM (200 mL) was added Boc₂O (18.1 g, 83.2 mmol) dropwise at 0-5° C. and then stirred for 3 h at the same temperature till (Boc)₂O was all consumed. The reaction mixture was washed with water (100 mL), the aqueous layer was extracted with DCM (50 mL), the combined organic phase was dried over anhydrous Na₂SO₄ and filtered. The filtrate was concentrated to dryness under reduced pressure to give A19-2X (12.1 g, yield: 90.8%).

### Step 2

The mixture of A19-2X (12.0 g, 74.9 mmol), A1-8A (15.8 g, 74.9 mmol), DIEA (48.5 g, 374.9 mmol) and HATU (34.3 g, 90.1 mmol) in DMF (100 mL) was stirred at room temperature for 15 min and then concentrated to dryness under reduced pressure. The residua was dissolved in DCM (100 mL), washed with water and brine, dried over anhydrous Na₂SO₄ and filtered. The filtrate was concentrated to dryness under reduced pressure to give A19-3X (16.3 g, yield: 61.6%). MS(ESI)(m/z): 354 ([M+H]⁺).

### Step 3

The mixture of A19-3X (16.0 g, 45.3 mmol) in DCM (160 mL) and TFA (32 g, 0.28 mol) was stir at room temperature for 1 h and then concentrated to dryness under reduced pressure to give A19-4X (10.9 g, yield: 95.6%).

### 16. Preparation of Compound A19

Following the general procedures of compound A20, replaced A1-9X with A1-10X, replaced A20-6 with A19-4X:

| | |
|---|---|
| A1-10X | 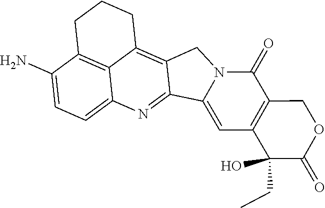 [SMILES: CC[C@@]1(O)C(=O)OCc2c1cc1n(c2=O)Cc2c-1nc1ccc(N)c3c1c2CCC3] |
| A19-4X | 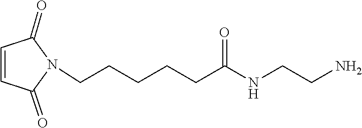 [SMILES: NCCNC(=O)CCCCCN1C(=O)C=CC1=O] |

### 17. Preparation of Compound A21

Following the general procedures of compound A20, replaced A20-6 with A19-4X:

| | |
|---|---|
| A19-4X | 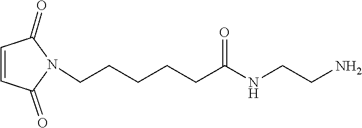 [SMILES: NCCNC(=O)CCCCCN1C(=O)C=CC1=O] |

### 18. Preparation of Compound A22

Following the general procedures of compound A20, replaced A20-1 with 3-(2-aminoethoxy) propanoic acid:

| | |
|---|---|
| 3-(2-aminoethoxy) propanoic acid | 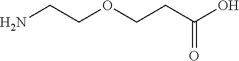 |

### 19. Preparation of Compound A23

Following the general procedures of compound A20, replaced A1-9X with A1-10X, replaced A20-1 with 3-(2-aminoethoxy) propanoic acid:

| | |
|---|---|
| A1-10X | 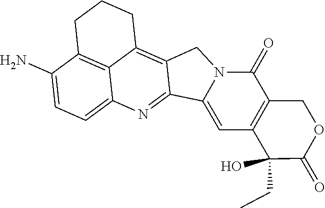 [SMILES: CC[C@@]1(O)C(=O)OCc2c1cc1n(c2=O)Cc2c-1nc1ccc(N)c3c1c2CCC3] |
| 3-(2-aminoethoxy) propanoic acid | 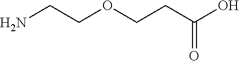 |

### 20. Preparation of Compound A24

Following the general procedures of compound A20, replaced A20-1 with 3-(2-(2-(2-aminoethoxy) ethoxy) ethoxy) propanoic acid:

| | |
|---|---|
| 3-(2-(2-(2- aminoethoxy) ethoxy)ethoxy) propanoic acid | 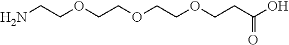 [SMILES: NCCOCCOCCOCCC(=O)O] |

### 21. Preparation of Compound A25

Following the general procedures of compound A20, replaced A20-1 with 3-(2-(2-(2-aminoethoxy) ethoxy) ethoxy) propanoic acid, replaced A1-9X with A1-10X:

| | |
|---|---|
| 3-(2- (2-(2- amino- | 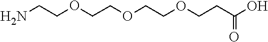 [SMILES: NCCOCCOCCOCCC(=O)O] |
| ethoxy) | |
| ethoxy) | |
| ethoxy) | |
| propanoic | |
| acid | |
| A1-10X | 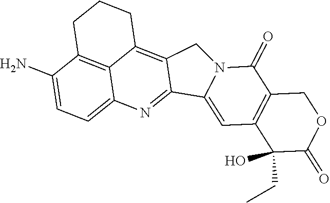 [SMILES: CC[C@@]1(O)C(=O)OCc2c1cc1n(c2=O)Cc2c-1nc1ccc(N)c3c1c2CCC3] |

### Example 2. Preparation of Antibody Conjugates

Replaced the HER2 targeted antibody Trastuzumab with 50 mM PB/1.0 mM EDTA buffer (pH 7.0) using a G25 desalination column, added TECP (8 eq.), stirred at 37° C. for 2 h to fully break the disulfide bonds between antibody chains, then adjusted the pH of the reduced antibody solution to 6.0 using phosphoric acid and cooled the water bath temperature to 25° C. for coupling reaction. The linker drug conjugate and GGFG Dxd (control compound) prepared by the method described in Example 1 was dissolved in DMA, and the linker drug conjugate (12 eq.) was added dropwise to the reduced antibody solution. DMA will be added until the final concentration is 10% (V/V), and the reaction mixture was stirred at 25° C. for 0.5 h to complete the reaction, and then the sample was filtered using a 0.22 um membrane and purified by a tangential flow ultrafiltration system to remove excess conjugated small molecules. The buffer solution is 50 mM PB/1.0 mM EDTA solution (pH=6.0). After purification, add a final concentration of 6% sucrose and store at −20° C. in a refrigerator. Measure the absorbance values at 280 nm and 370 nm using UV method, and calculate the DAR value. Most of the linker drug conjugates in this technical solution did not produce precipitation during the coupling process, and the DAR values of the conjugates were 6-8. The DAR values were obtained using HIC-HPLC, The determination was carried out by RP-HPLC or LCMS, and the polymer ratio of the conjugate was detected to be within the normal range by SEC-HPLC, indicating that the antibody drug conjugate of the present invention has good solubility and drug properties, and no precipitation occurs during the coupling process.

### Example 3. Cytotoxicity Test In Vitro

Stable transfection of high expression Her2, high expression SK-BR-3 and BT-474 human breast cancer cells, NCI-N87 human gastric cancer cells and human non-small cell lung cancer cell line A549 were selected as the cell lines for in vitro activity detection in this experiment, and the killing dose and effect of different antibody coupled drugs on cells were observed. Preliminary selection of plate density for each cell type: 2×10³ cells/well, followed by cytotoxicity assay after 16-24 h; Next, the antibody coupled drug prepared in Example 2 was tested with a final concentration set at 5000 nM as the starting concentration, and a series of 10 concentrations (4-10 fold dilution) were designed for 5000-0.006 nM. The killing (or inhibition) changes were observed for 120 hours, and chemiluminescence staining (Luminescent Cell Viability Assay) was performed. The IC50 was calculated after reading the fluorescence data.

The linkers in the present invention are all cleavable linkers. Therefore, the released camptothecin analogs were first subjected to in vitro cytotoxic activity testing, and the results are shown in the table below. The test results showed that most of the compounds had better cell killing activity than the control compound D×d.

| Name | structure | IC₅₀ (A549, nM) |
|---|---|---|
| Dxd | 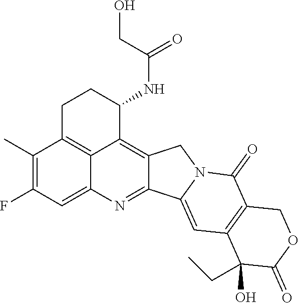 [SMILES: CC[C@@]1(O)C(=O)OCc2c1cc1n(c2=O)Cc2c-1nc1cc(F)c(C)c3c1c2[C@@H](NC(=O)CO)CC3] | 45.1 |
| A16-FD | 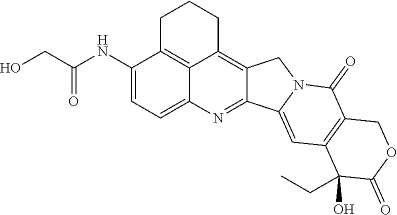 [SMILES: CC[C@@]1(O)C(=O)OCc2c1cc1n(c2=O)Cc2c-1nc1ccc(NC(=O)CO)c3c1c2CCC3] | 79.5 |
| A1-FD | 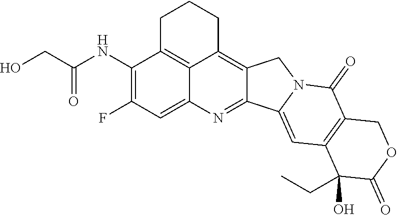 [SMILES: CC[C@@]1(O)C(=O)OCc2c1cc1n(c2=O)Cc2c-1nc1cc(F)c(NC(=O)CO)c3c1c2CCC3] | 69.1 |
| A9-FD | 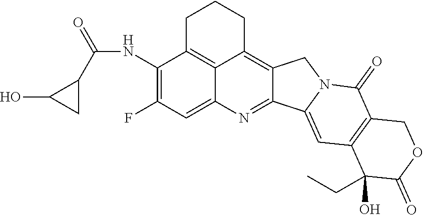 [SMILES: CC[C@@]1(O)C(=O)OCc2c1cc1n(c2=O)Cc2c-1nc1cc(F)c(NC(=O)C3CC3O)c3c1c2CCC3] | 25.2 |
| A11-FD | 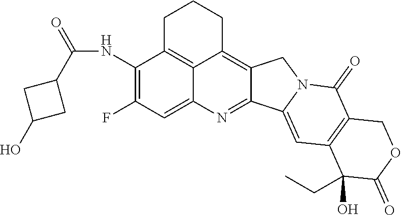 [SMILES: CC[C@@]1(O)C(=O)OCc2c1cc1n(c2=O)Cc2c-1nc1cc(F)c(NC(=O)C3CC(O)C3)c3c1c2CCC3] | 2.1 |
| A2-FD | 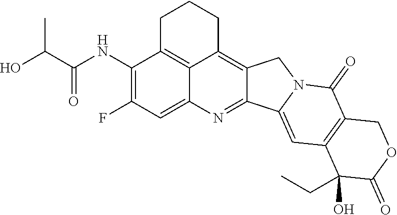 [SMILES: CC[C@@]1(O)C(=O)OCc2c1cc1n(c2=O)Cc2c-1nc1cc(F)c(NC(=O)C(C)O)c3c1c2CCC3] | 0.53 |
| A3-FD | 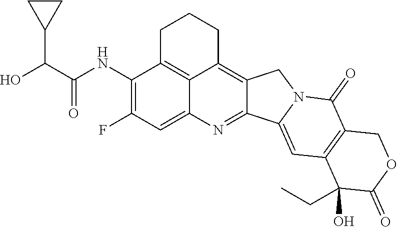 [SMILES: CC[C@@]1(O)C(=O)OCc2c1cc1n(c2=O)Cc2c-1nc1cc(F)c(NC(=O)C(O)C3CC3)c3c1c2CCC3] | 5.5 |

Further, cytotoxic activity testing in vitro of the coupled ADC drugs was conducted. The activity test results showed the ADCs prepared from the compounds in this application all exhibited certain anti-tumor activity, IC₅₀ ranged from 10⁻⁶˜10⁻¹⁰ M, significantly stronger anti-tumor activity compared to the control sample.

### Example 4. Anti-Tumor Efficacy Testing In Vivo

Measure the efficacy of the combination of the present invention in vivo, which involves implanting cancer cells into homologous or heterologous transplants in rodents and treating tumors with the combination. Treat the test mice with drugs or controls and monitor for several weeks or longer to measure the time to tumor doubling, logarithmic cell killing, and tumor inhibition.

### 1) Experimental Animals

BALB/cA nude mice, 6-7 weeks old, female, purchased from Shanghai Lingchang Biotechnology Co., Ltd.

### 2) Experimental Operation

Naked mice were subcutaneously inoculated with human gastric cancer NCI-N87 cells. After the tumor grew to 100-250 mm³, the animals were randomly divided into groups (DO). Measure tumor volume 2-3 times per week, weigh the mice, and record the data. The formula for calculating tumor volume (V) is: V=1/2×a×b², where a and b represent length and width, respectively. T/C (%)=(T−T₀)/(C−C₀)×100%, where T and C are the tumor volumes at the end of the experiment for the experimental group and the control group, respectively; T₀ and C₀ were the tumor volumes at the beginning of the experiment for the experimental group and the control group, respectively.

From the activity test results, the ADC prepared from the compounds in this application exhibited certain in vivo anti-tumor activity, and had significantly stronger anti-tumor activity compared to the control sample. Tumor bearing mice can tolerate the above drugs well without experiencing symptoms such as weight loss.

### INDUSTRIAL APPLICABILITY

The inhibitor of the present invention is compound A or its tautomer, mesomer, racemate, optical antipode, diastereoisomer, or a mixture form thereof, or a pharmaceutically acceptable salt thereof; the compound may also be further prepared to obtain an antibody drug conjugate, the antibody drug conjugate has good solubility and pharmaceutical properties, and the conjugation process does not result in precipitation, the antibody drug conjugate exhibits obvious in-vivo anti-tumor activity, and shows markedly stronger anti-tumor activity when compared with a control sample.

The above detailed description is provided by way of explanation and examples, and is not intended to limit the scope of the appended claims. The various variations of the embodiments listed in this application are obvious to those skilled in the art and are within the scope of the appended claims and their equivalents.

1. An inhibitor compound or its tautomer, mesomer, racemate, optical antipode, diastereoisomer, or the mixture form thereof, or the pharmaceutically acceptable salt thereof, specifically, it comprises the structure shown in formula (A): 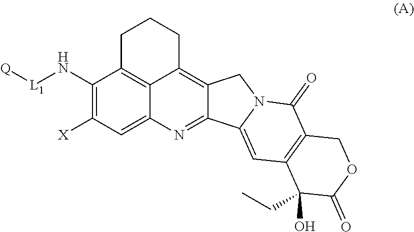
Wherein X is hydrogen and fluorine; Q is hydrogen or a group that can be conjugated with antibodies, L1 is a group that connects Q and the amino group of the drug.

2. The compound according to claim 1, or its tautomer, mesomer, racemate, optical antipode, diastereoisomer, or the mixture form thereof, or the pharmaceutically acceptable salt thereof, specifically, its Q part is a group that can be conjugated with the thiol group on the antibody, selected from maleimide.

3. The compound according to claim 1, or its tautomer, mesomer, racemate, optical antipode, diastereoisomer, or the mixture form thereof, or the pharmaceutically acceptable salt thereof, wherein L1 is a group that connects Q and the amino group of the drug, selected from 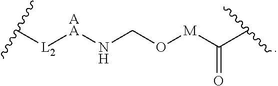
Wherein L2 is an optionally substituted C3-C7 alkylene, C3-C8 cyclic alkyl, optionally substituted diethylene glycol to octaethylene glycol acyl; AA is a peptide fragment composed of 2 to 4 amino acids; M is a methylene, C1-C6 alkyl, or cycloalkyl substituted methylene, trifluoromethyl substituted methylene, and C3-C6 cyclic alkyl.

4. The compound according to claim 3, or its tautomer, mesomer, racemate, optical antipode, diastereoisomer, or the mixture form thereof, or the pharmaceutically acceptable salt thereof, wherein L1 is a group that connects Q and the amino group of the drug, selected from 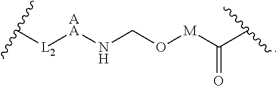
Wherein AA is a peptide fragment selected from ^{NH}-Phe-Lys-^{C═O}, ^{NH}-Val-Cit-^{C═O}, ^{NH}-Val-Ala-^{C═O}, ^{NH}_Phe-Cit-^{C═O}, ^{NH}-Gly-Val-^{C═O}, ^{NH}-Ala-Lys-^{C═O}, ^{NH}-Ala-Ala-Ala-^{C═O}, ^{NH}-Glu-Val-Ala-^{C═O}, ^{NH}-Glu-Val-Cit-^{C═O}, ^{NH}-Gly-Gly-Phe-Gly-^{C═O}.

5. The compound according to claim 1, or its tautomer, mesomer, racemate, optical antipode, diastereoisomer, or the mixture form thereof, or the pharmaceutically acceptable salt thereof, wherein L1 is a group that connects Q and the amino group of the drug, selected from 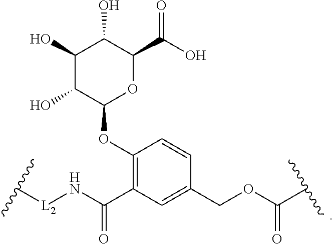
Wherein L2 is an optionally substituted C3-C7 alkylene, C3-C8 cyclic alkyl, optionally substituted diethylene glycol to octaethylene glycol acyl.

6. The compound according to claim 1, characterized in that the specific molecular structure of formula A is as follows:

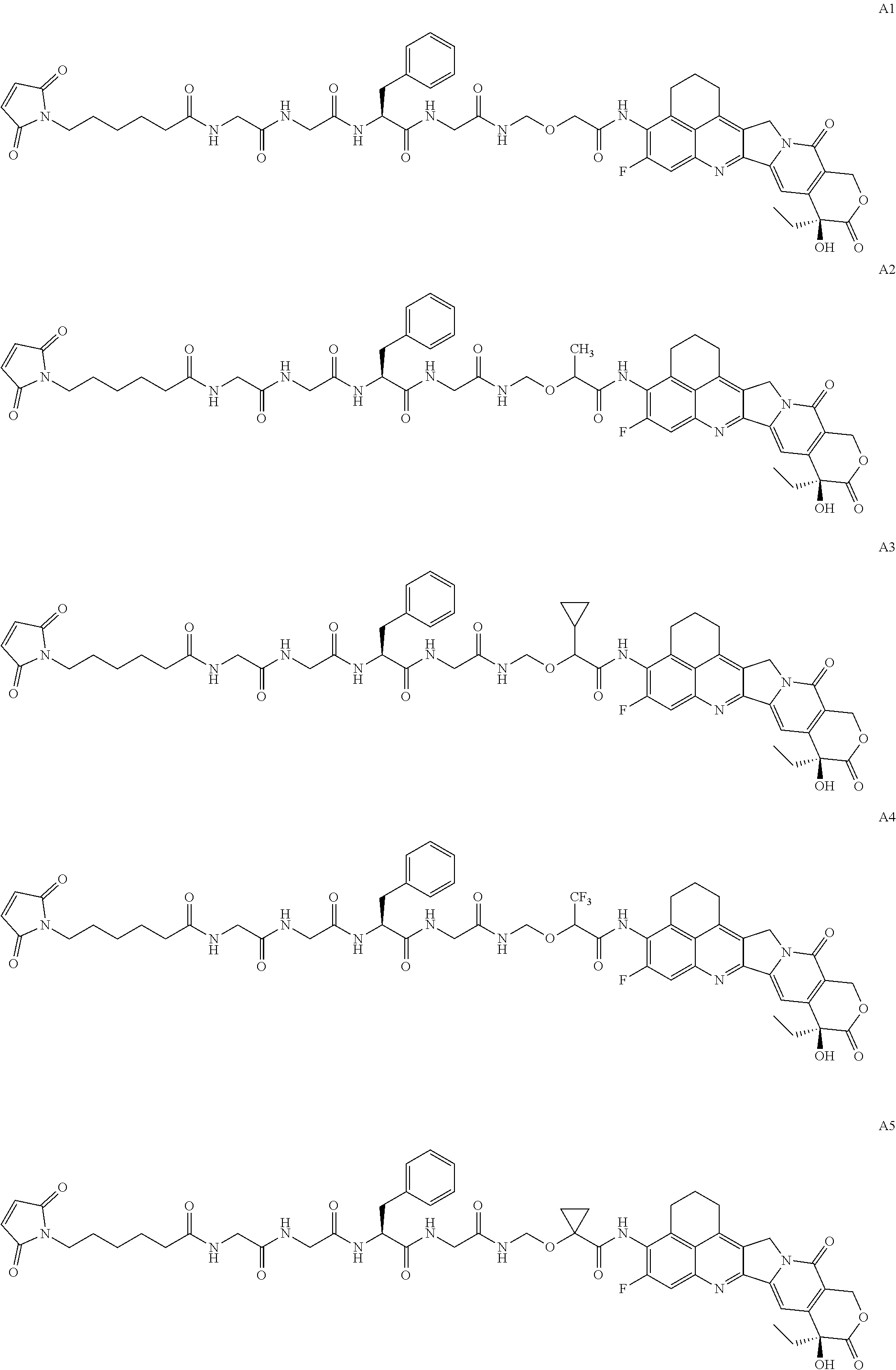

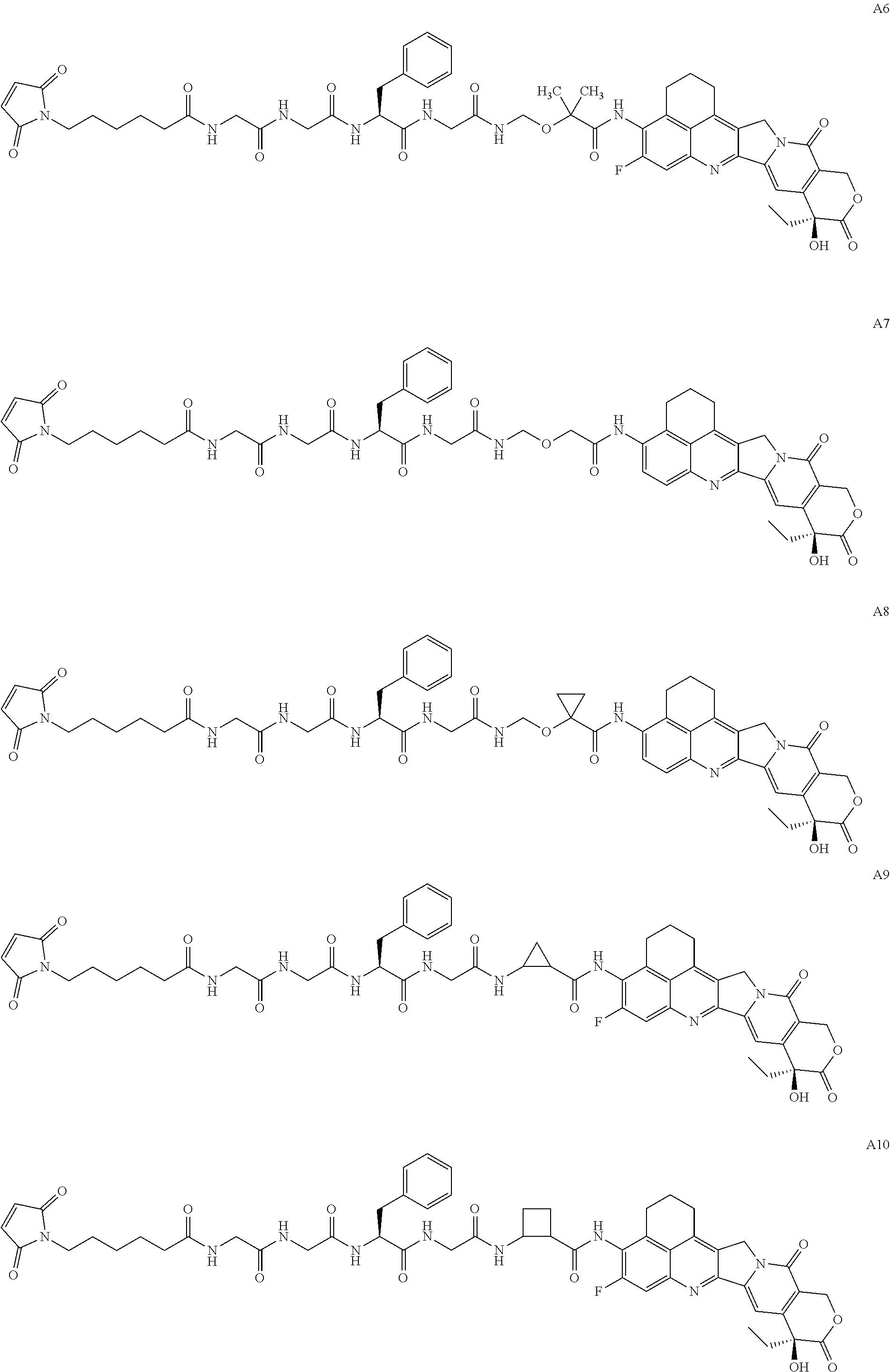

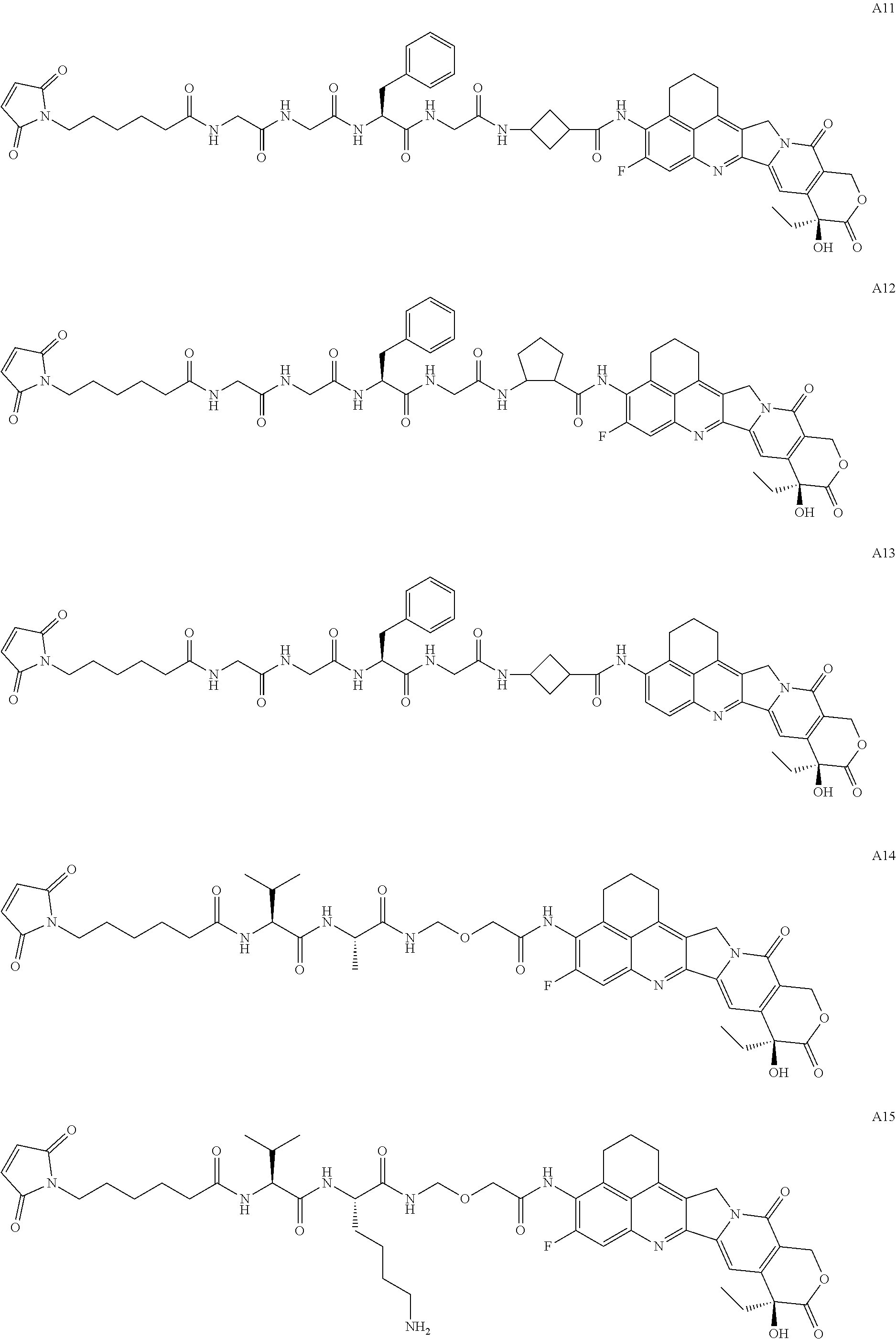

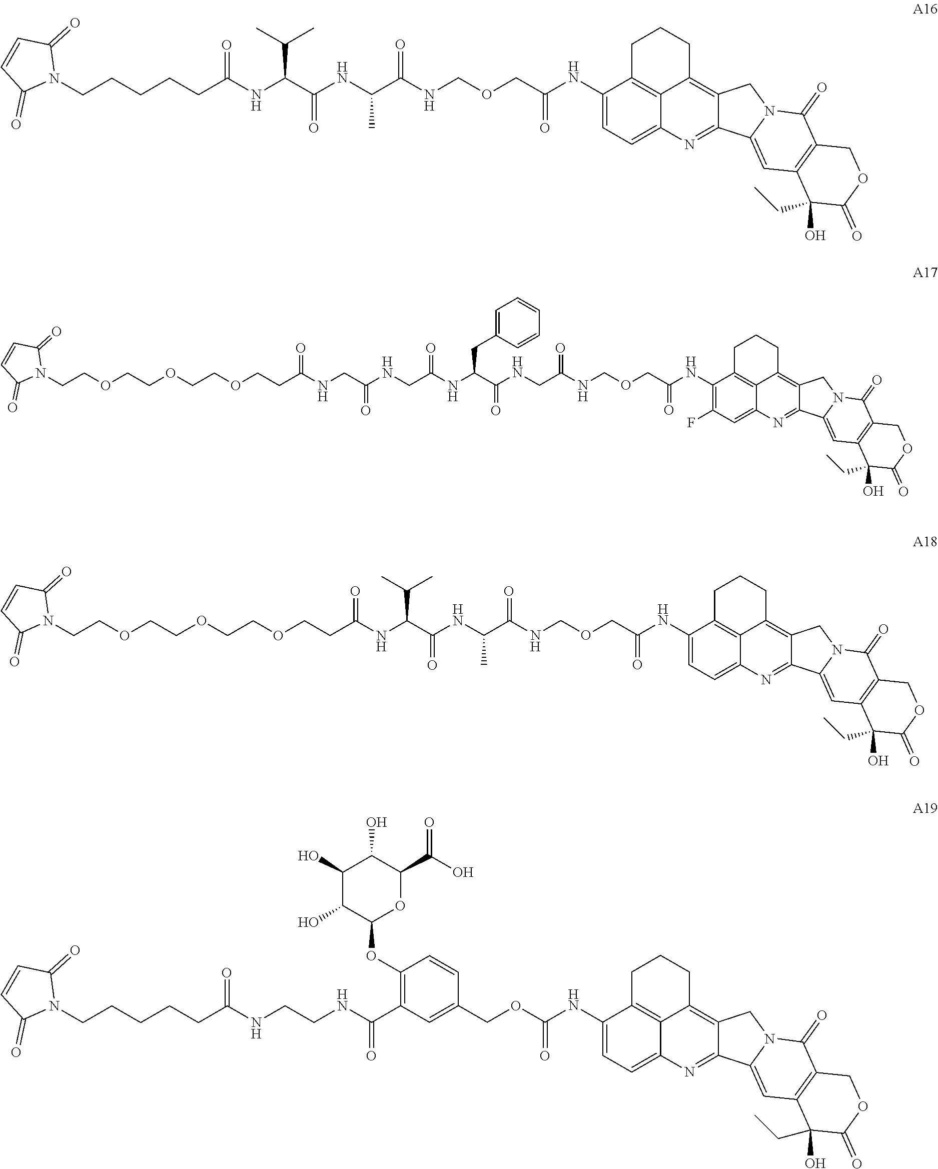

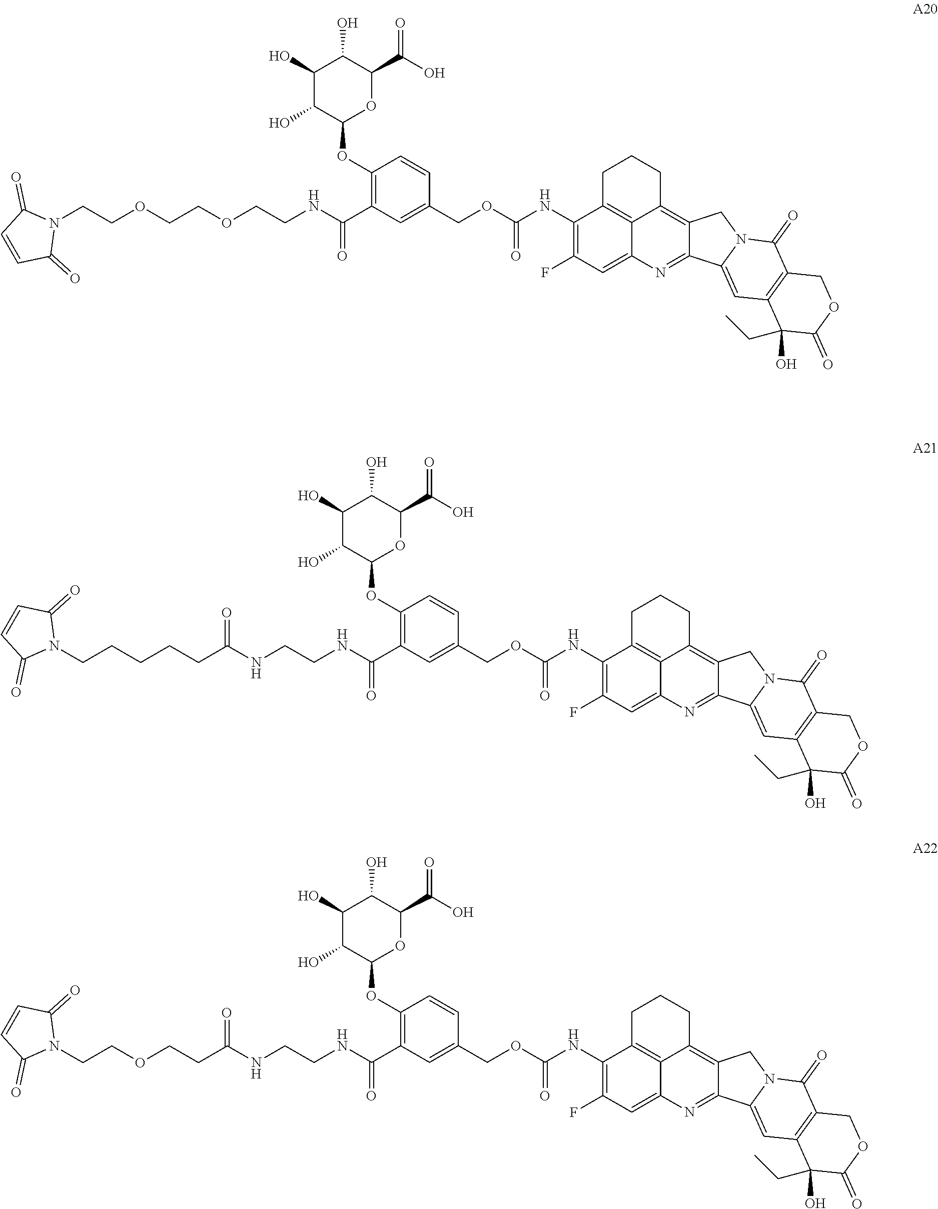 [SMILES: CC[C@@]1(O)C(=O)OCc2c1cc1n(c2=O)Cc2c-1nc1cc(F)c(NC(=O)OCc3ccc(O[C@@H]4O[C@H](C(=O)O)[C@@H](O)[C@H](O)[C@H]4O)c(C(=O)NCCOCCOCCN4C(=O)C=CC4=O)c3)c3c1c2CCC3 ; CC[C@@]1(O)C(=O)OCc2c1cc1n(c2=O)Cc2c-1nc1cc(F)c(NC(=O)OCc3ccc(O[C@@H]4O[C@H](C(=O)O)[C@@H](O)[C@H](O)[C@H]4O)c(C(=O)NCCNC(=O)CCCCCN4C(=O)C=CC4=O)c3)c3c1c2CCC3 ; CC[C@@]1(O)C(=O)OCc2c1cc1n(c2=O)Cc2c-1nc1cc(F)c(NC(=O)OCc3ccc(O[C@@H]4O[C@H](C(=O)O)[C@@H](O)[C@H](O)[C@H]4O)c(C(=O)NCCNC(=O)CCOCCN4C(=O)C=CC4=O)c3)c3c1c2CCC3]

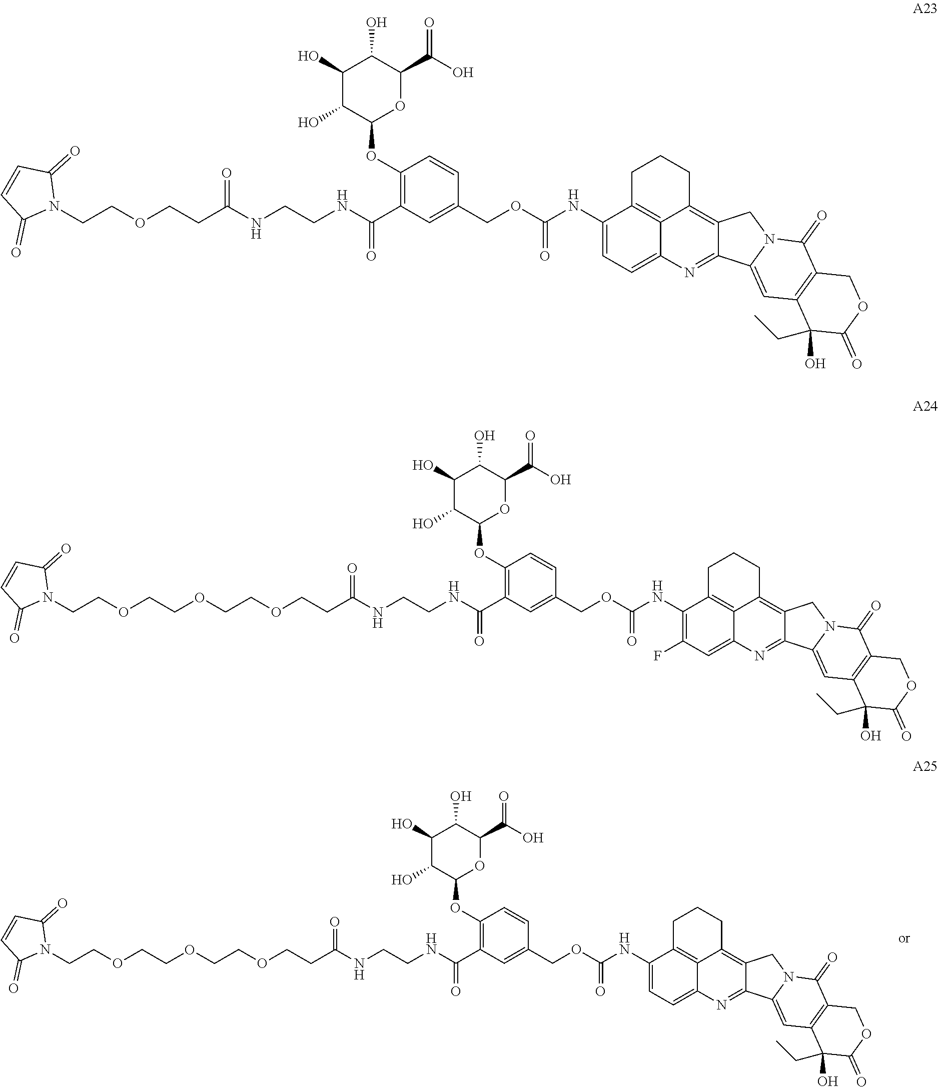 [SMILES: CC[C@@]1(O)C(=O)OCc2c1cc1n(c2=O)Cc2c-1nc1ccc(NC(=O)OCc3ccc(O[C@@H]4O[C@H](C(=O)O)[C@@H](O)[C@H](O)[C@H]4O)c(C(=O)NCCNC(=O)CCOCCN4C(=O)C=CC4=O)c3)c3c1c2CCC3 ; CC[C@@]1(O)C(=O)OCc2c1cc1n(c2=O)Cc2c-1nc1cc(F)c(NC(=O)OCc3ccc(O[C@@H]4O[C@H](C(=O)O)[C@@H](O)[C@H](O)[C@H]4O)c(C(=O)NCCNC(=O)CCOCCOCCOCCN4C(=O)C=CC4=O)c3)c3c1c2CCC3 ; CC[C@@]1(O)C(=O)OCc2c1cc1n(c2=O)Cc2c-1nc1ccc(NC(=O)OCc3ccc(O[C@@H]4O[C@H](C(=O)O)[C@@H](O)[C@H](O)[C@H]4O)c(C(=O)NCCNC(=O)CCOCCOCCOCCN4C(=O)C=CC4=O)c3)c3c1c2CCC3]

7. An antibody drug conjugate or its tautomer, mesomer, racemate, optical antipode, diastereoisomer, or the mixture form thereof, or the pharmaceutically acceptable salt thereof, specifically, it comprises the structure shown in formula (B): 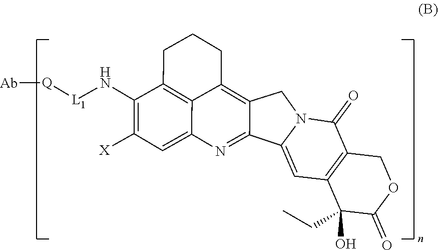
Wherein X is hydrogen and fluorine; Q is a group that can be conjugated with antibodies, L1 is a group that connects Q and the amino group of the drug, Ab is a ligand, n=1-8.

8. The antibody drug conjugate according to claim 7, or its tautomer, mesomer, racemate, optical antipode, diastereoisomer, or the mixture form thereof, or the pharmaceutically acceptable salt thereof, its Q part comprises the conjugated compound which is obtained by conjugating with the thiol group, selected from

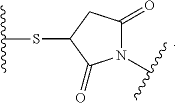 [SMILES: *SC1CC(=O)N(*)C1=O]

9. The antibody drug conjugate according to claim 7, or its tautomer, mesomer, racemate, optical antipode, diastereoisomer, or the mixture form thereof, or the pharmaceutically acceptable salt thereof, wherein L1 is a group that connects Q and the amino group of the drug, selected from 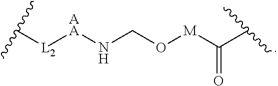
Wherein L2 is an optionally substituted C3-C7 alkylene, C3-C8 cyclic alkyl, optionally substituted diethylene glycol to octaethylene glycol acyl; AA is a peptide fragment composed of 2 to 4 amino acids; M is a methylene, C1-C6 alkyl, or cycloalkyl substituted methylene, trifluoromethyl substituted methylene, and C3-C6 cyclic alkyl.

10. The antibody drug conjugate according to claim 9, or its tautomer, mesomer, racemate, optical antipode, diastereoisomer, or the mixture form thereof, or the pharmaceutically acceptable salt thereof, wherein L1 is a group that connects Q and the amino group of the drug, selected from 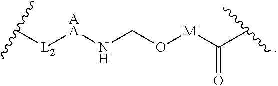
Wherein AA is a peptide fragment selected from ^{NH}-Phe-Lys-^{C═O}, ^{NH}-Val-Cit-^{C═O}, ^{NH}-Val-Ala-^{C═O}, ^{NH}-Phe-Cit-^{C═O}, ^{NH}-Gly-Val-^{C═O}, ^{NH}-Ala-Lys-^{C═O}, ^{NH}-Ala-Ala-Ala-^{C═O}, ^{NH}-Glu-Val-Ala-^{C═O}, ^{NH}-Glu-Val-Cit-^{C═O}, ^{NH}-Gly-Gly-Phe-Gly-^{C═O}.

11. The antibody drug conjugate according to claim 7, or its tautomer, mesomer, racemate, optical antipode, diastereoisomer, or the mixture form thereof, or the pharmaceutically acceptable salt thereof, wherein L1 is a group that connects Q and the amino group of the drug, selected from 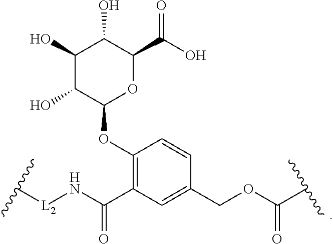
Wherein L2 is an optionally substituted C3-C7 alkylene, C3-C8 cyclic alkyl, optionally substituted diethylene glycol to octaethylene glycol acyl;

12. The compound according to claim 7, characterized in that the specific molecular structure of formula B is as follows: 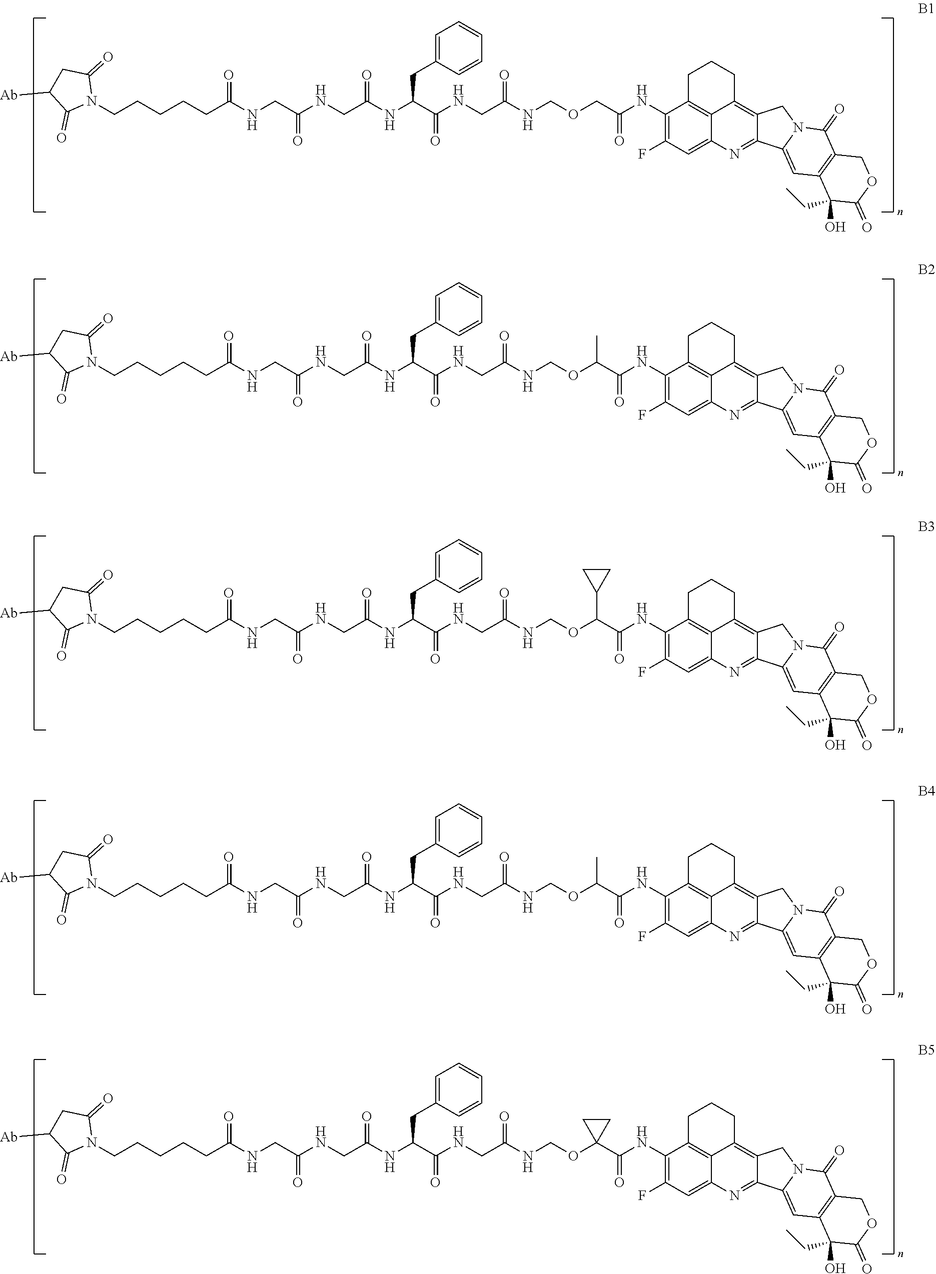 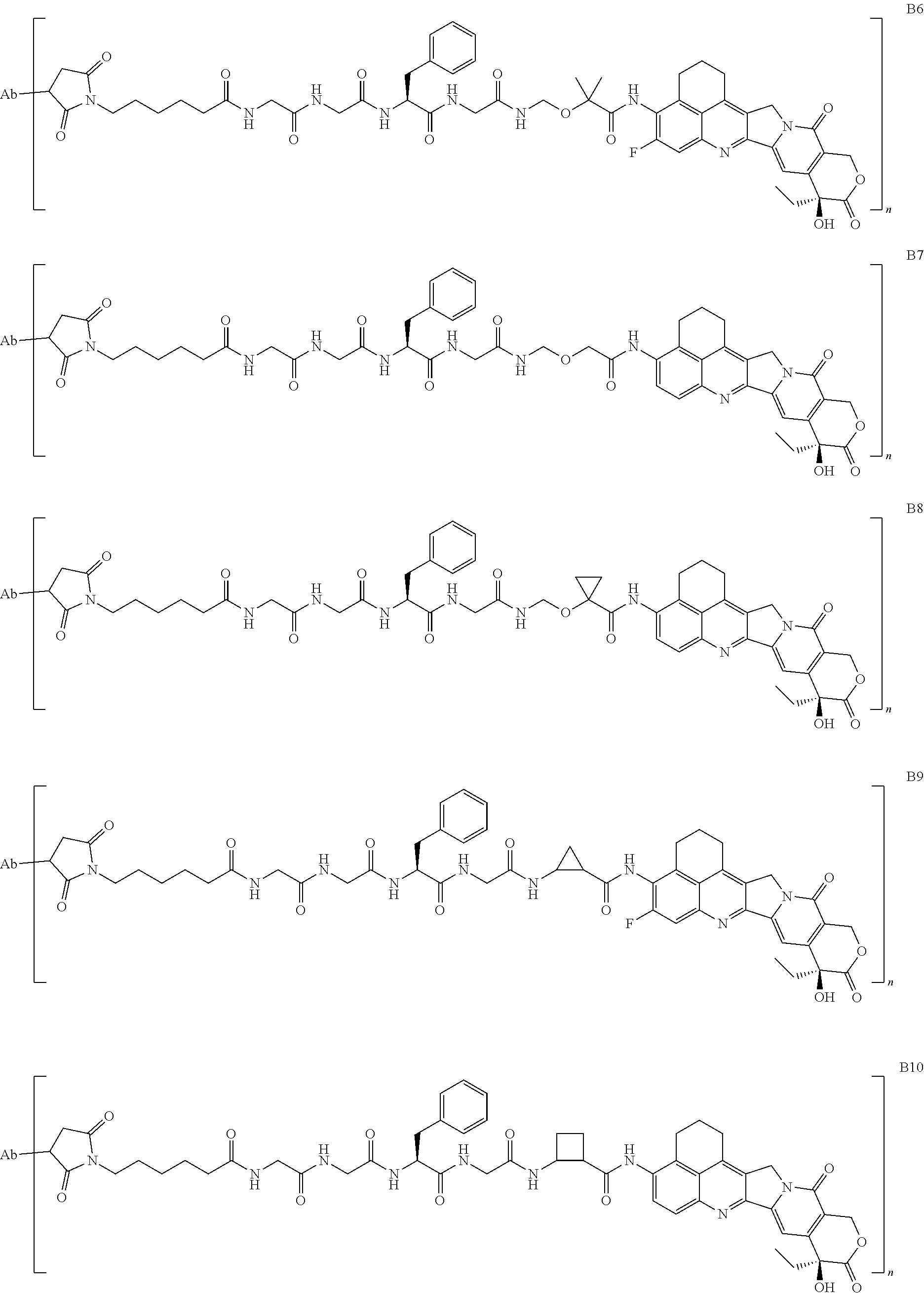 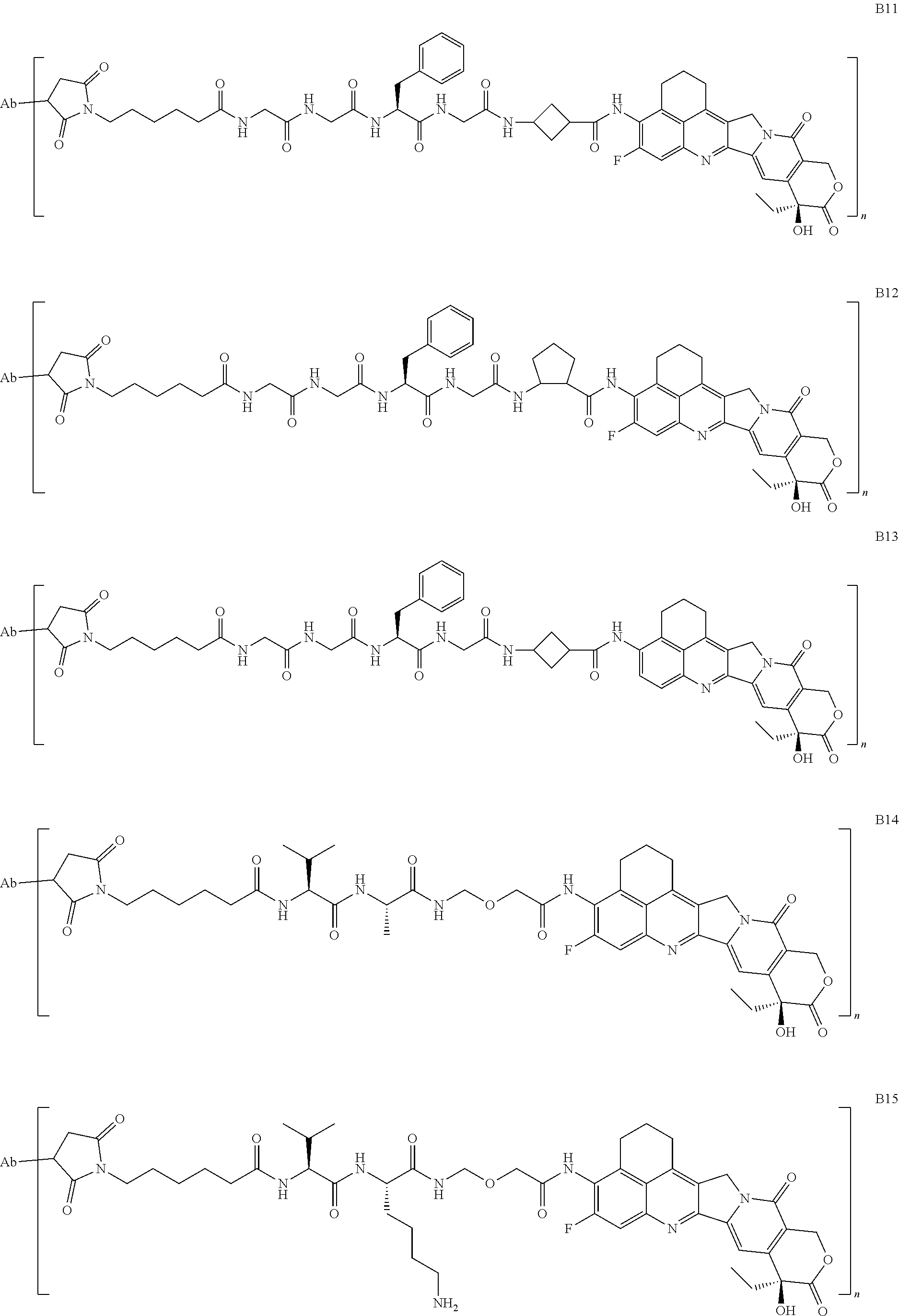 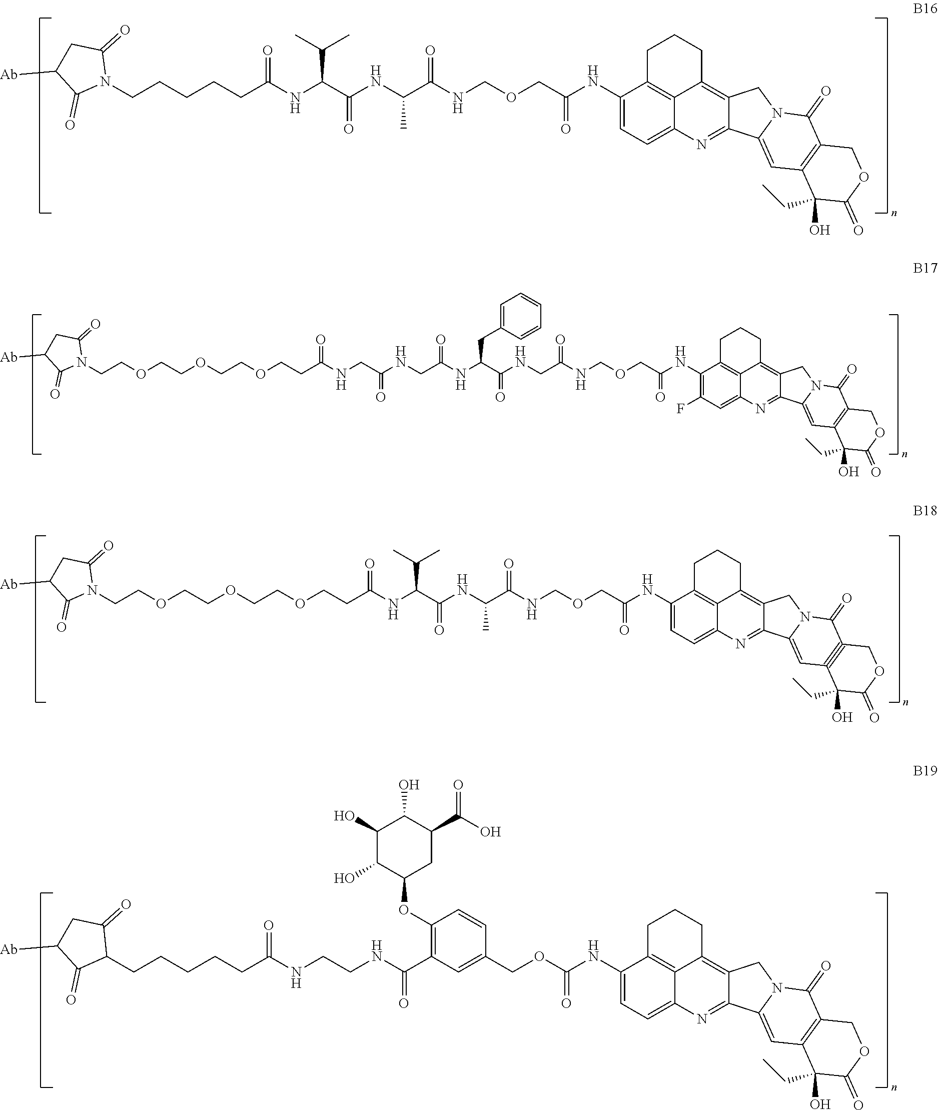 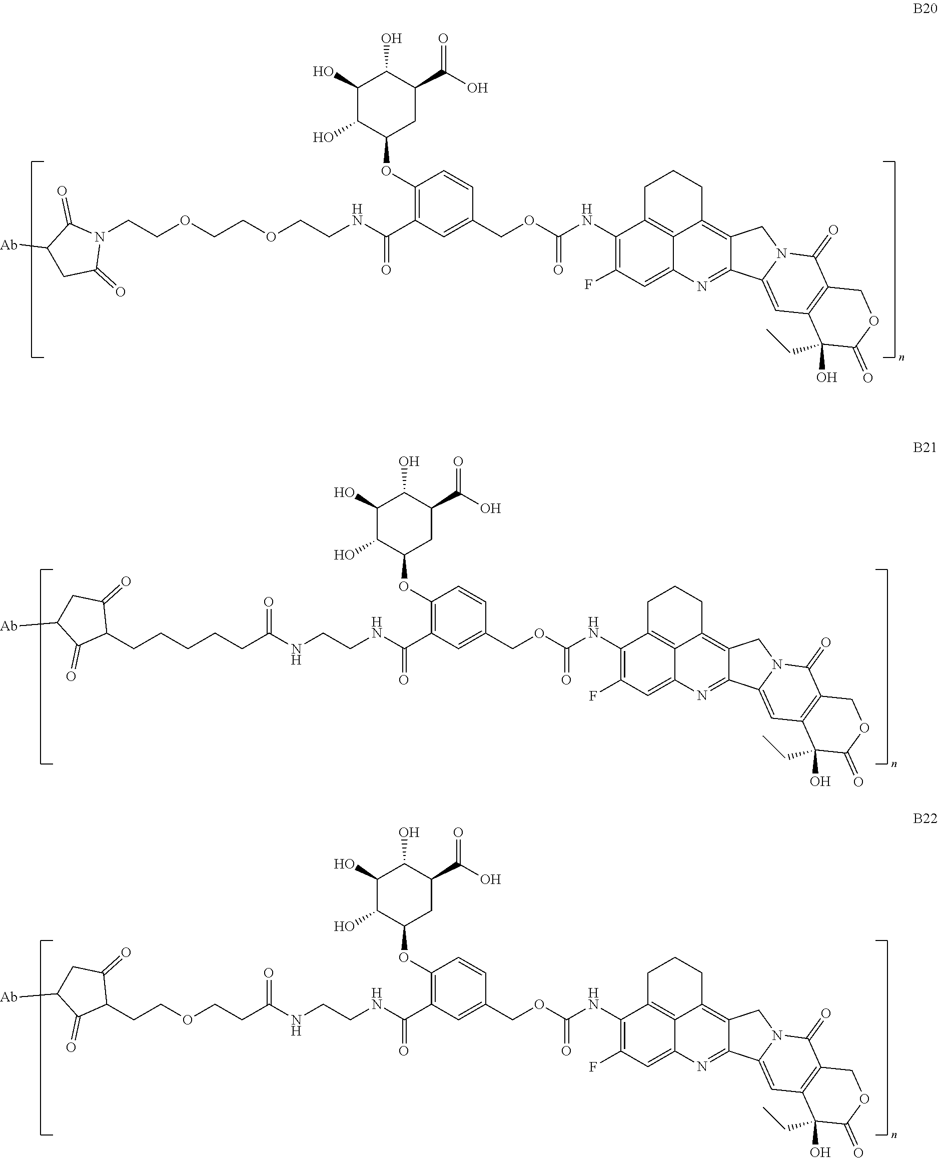 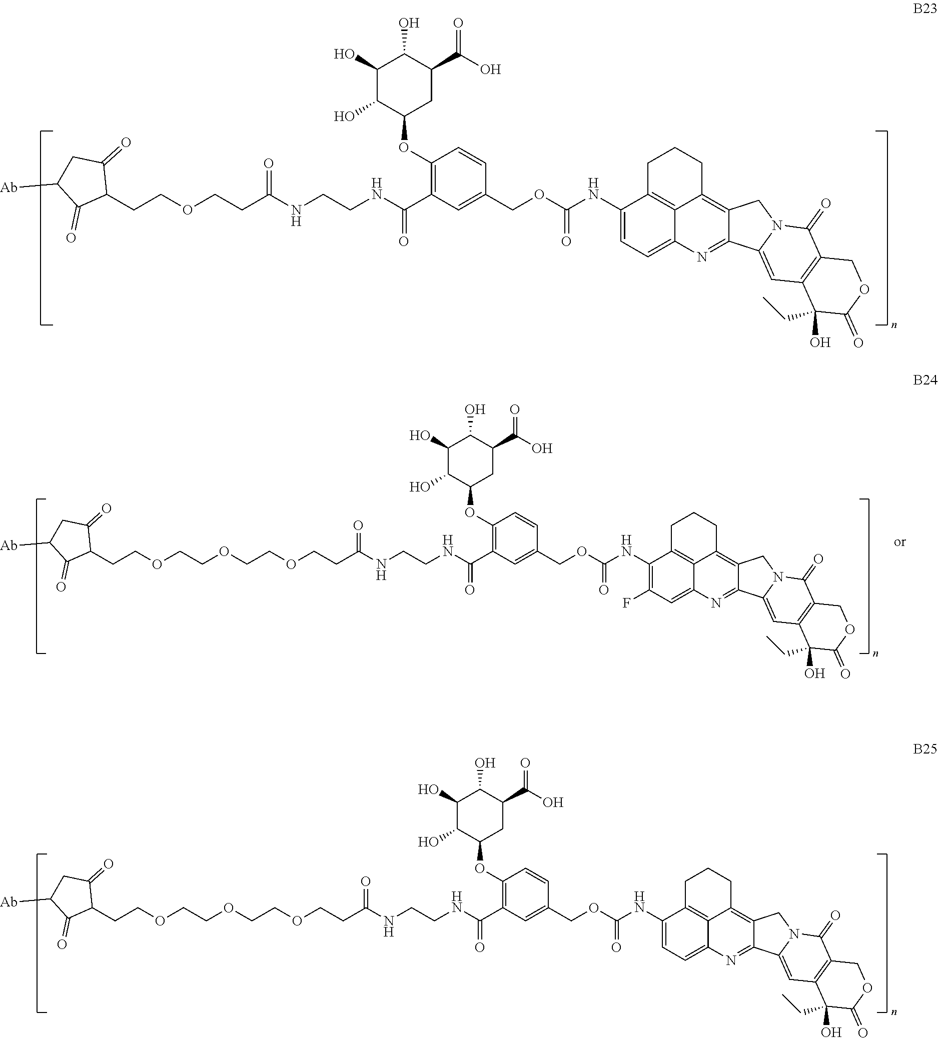
Wherein Ab is a ligand, n=1-8.

13. The antibody drug conjugate according to claim 12 or its tautomer, mesomer, racemate, optical antipode, diastereoisomer, or the mixture form thereof, or the pharmaceutically acceptable salt thereof, wherein Ab is selected from mouse derived antibodies, chimeric antibodies, humanized antibodies, or fully humanized antibodies.

14. The antibody drug conjugate according to claim 13, or its tautomer, mesomer, racemate, optical antipode, diastereoisomer, or the mixture form thereof, or the pharmaceutically acceptable salt thereof, wherein Ab comprises monoclonal antibodies.

15. The antibody drug conjugate according to claim 14, or its tautomer, mesomer, racemate, optical antipode, diastereoisomer, or the mixture form thereof, or the pharmaceutically acceptable salt thereof, wherein Ab comprises bispecific antibodies.

16. The antibody drug conjugate according to claim 15, wherein the according antibody can bind to HER2, HER3, CD19, CD20, CD22, CD30, CD33, CD37, CD45, CD56, CD66e, CD70, CD74, CD79b, CD138, CD147, CD223, EpCAM, Mucin 1, STEAP1, GPNMB, FGF2, FOLR1, EGFR, EGFRvIII, Tissue factor, c-MET, FGFR, Nectin 4, AGS-16, Guanylyl cyclase C, Mesothelin, SLC44A4, PSMA, EphA2, AGS-5, GPC-3, c-KIT, ROR1, PD-L1, CD27L, 5T4, Mucin 16, NaPi2b, STEAP, SLITRK6, ETBR, BCMA, Trop-2, CEACAM5, SC-16, SLC39A6, Delta like protein 3, or Claudin 18.2 tumor associated antigens.

17. A pharmaceutical composition comprises: (a) an antibody drug conjugate according to claim 7; And (b) pharmaceutically acceptable diluents, carriers, or excipients.

18. The use of antibody drug conjugates according to claim 7 for Preparation of therapeutic drugs for tumors.

19. Preparation method of antibody drug conjugates according to claim 7 comprising the following steps:
a. reacting antibodies with reducing reagents in buffer solution to obtain reduced antibodies;
b. the linker-payload conjugate (A) is conjugated in a mixture of buffer solution and organic solvent with the reduced antibody obtained in the step a to obtain the antibody drug conjugate.
